(19) United States
(12) Patent Application Publication
(10) Pub. No.: US 2024/0327792 A1
(43) Pub. Date: Oct. 3, 2024

(54) FLUID SHEAR STRESS FOR EX VIVO ACTIVATION OF IMMUNE EFFECTOR CELLS

(71) Applicant: Vanderbilt University, Nashville, TN (US)

(72) Inventors: Michael R. King, Nashville, TN (US); Jacob M. Hope, Nashville, TN (US); Jenna A. Dombroski, Nashville, TN (US); Nicole Suzanne Sarna, Nashville, TN (US)

(21) Appl. No.: 18/575,437

(22) PCT Filed: Jun. 17, 2022
(86) PCT No.: PCT/US2022/073015

§ 371 Date: Dec. 29, 2023

Related U.S. Application Data
(60) Provisional application No. 63/218,557, filed on Jul. 6, 2021.

(51) Int. Cl. C12N 5/0784; C12N 5/0783; C12N 13/00

(52) U.S. Cl. CPC C12N 5/0639; C12N 5/0636; C12N 13/00; C12N 2501/51; C12N 2501/515

(57) ABSTRACT

Disclosed herein is an improved method for ex vivo activation of immune effector cells that involves applying an effective amount of fluid shear stress to the immune effector cells, and exposing the immune effector cells to one or more activating agents before, during, or after shear stress application. Also disclosed is a method for manufacturing a human immune effector cell therapeutic that involves obtaining a population of peripheral blood mononuclear cells (PBMCs) that comprises immune effector cells in a fluid medium; applying fluid shear stress to the immune effector cells, optionally exposing the immune IC effector cells to one or more activating agents before, during, or after shear stress application; and culturing the activated immune effector cells in a cell growth medium to expand the activated immune effector cells, thereby manufacturing the human immune effector cell therapeutic.

### CROSS-REFERENCE TO RELATED APPLICATIONS

This application claims benefit of U.S. Provisional Application No. 63/218,557, filed Jul. 6, 2021, which is hereby incorporated herein by reference in its entirety.

### BACKGROUND

Adoptive T cell therapies, such as chimeric antigen receptor (CAR) T cells, are at the forefront of emerging cancer immunotherapy (Miliotou, A. N. et al. Curr. Pharm. Biotechnol. 2018 19:5-18; Feins, S. et al. Am. J. Hematol. 2019 94:S3-S9). In a clinical trial where CD19-targeting CAR T cells were used to treat B-cell acute lymphoblastic leukemia, 83% of treated patients showed complete remission after 29 months (Park, J. H. et al. N. Engl. J. Med. 2018 378:449-459). Despite these encouraging results, CAR T cells still suffer from certain limitations, as relapse rates remain high in CAR T cell treated patients (Park, J. H. et al. N. Engl. J. Med. 2018 378:449-459; Maude, S. L. et al. N. Engl. J. Med. 2018 378:439-448). However, promoting the persistence and proliferation of CAR T cells in vivo reduces the chance of relapse (Porter, D. L. et al. Sci. Transl. Med. 2015 7:303ra139). One key step in producing CAR T cells is the ex vivo activation of T cells isolated from the cancer patient. The method of ex vivo activation has been shown to have a substantial effect on the persistence of CAR T cells in vivo (Jafarzadeh, L. et al. Front. Immunol. 2020 11). This has motivated research into identifying improved methods of ex vivo T cell activation by using artificial antigen presenting cells, antibody-coated beads, exogenous cytokines, and molecular agonists (Torres Chavez, A. et al. J. Immunother. Cancer. 2019 7:330; Butler, M. O. et al. Clin. Cancer Res. 2007 13:1857-1867; Li, Y. et al. J. Transl. Med. 2010 8:104; Poltorak, M. P. et al. Sci. Rep. 2020 10:17832; Besser, M. J. et al. Cytotherapy. 2009 11:206-217; Liu, C. S. C. et al. J. Immunol. 2018 200:1255-1260).

### SUMMARY

Disclosed herein is an improved method for ex vivo activation of immune effector cells that involves applying an effective amount of fluid shear stress to the immune effector cells, and exposing the immune effector cells to one or more activating agents before, during, or after shear stress application. In some embodiments, the effective amount of fluid shear stress is from 0.5 dynes/cm² to 20 dynes/cm² fluid shear stress for from 5 minutes to 120 minutes.

Therefore, also disclosed is a method for manufacturing a human immune effector cell therapeutic that involves obtaining a population of peripheral blood mononuclear cells (PBMCs) that comprises immune effector cells in a fluid medium; applying from 0.5 dynes/cm² to 20 dynes/cm² fluid shear stress to the immune effector cells for from 5 minutes to 120 minutes, and optionally exposing the immune effector cells to one or more activating agents before, during, or after shear stress application; and culturing the activated immune effector cells in a cell growth medium to expand the activated immune effector cells, thereby manufacturing the human immune effector cell therapeutic.

In some embodiments, the immune effector cell is a T cell, such as a CAR-T cell. Therefore, in some embodiments, the one or more activating agents includes an anti-CD3 antibody and an anti-CD28 antibody. In some embodiments, the immune effector cell is a dendritic cell (DC). Therefore, in some embodiments, the one or more activating agents includes lipopolysaccharide (LPS). In some embodiments, the immune effector cell is a natural killer (NK) cell. Therefore, in some embodiments, the one or more activating agents includes interleukin-2 (IL-2).

The shear stress can be applied using any suitable flow device which is capable of inducing flow of the fluid medium. For example, the flow device can be a cone-and-plate device or a parallel plate flow device. In some embodiments, the device is a closed loop peristaltic pump system.

In some embodiments, the fluid medium comprises an additive to provide a viscosity of from 0.01 poise to 6.0 poise. For example, suitable additives include glycerol, pluronic F68, dextran, polyethylene glycol (PEG), or a combination thereof.

In some embodiments, the appropriate shear stress is determined based on Piezo1 expression by the immune effector cells. Therefore, in some embodiments, the method further involves assaying the immune effector cells for Piezo1 expression, and selecting a fluid shear stress, viscosity, time, or any combination thereof based on the Piezo1 expression levels.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1F show phosphorylation of ZAP70 by CD3/CD28 antibody and FSS treatment. FIG. 1A is a schematic of FSS treatment via cone-and-plate viscometer. G=shear rate, ω=angular velocity, θ=angle of cone, τ=fluid shear stress, μ=viscosity. FIG. 1B is a representative flow plot and graph of ZAP70 phosphorylation of Jurkat cells treated with or without FSS (N=3). FIG. 1C is a representative flow plot and graph of ZAP70 phosphorylation in Jurkat cells treated with or without FSS and CD3/CD28 antibodies (N=3). Untreated: Jurkat cells that were treated with neither FSS or antibodies; static: Jurkat cells treated with only antibodies; shear: cells treated with antibodies and FSS. FIG. 1D shows change in FRET efficiency of a ZAP70 targeted antibody and a CD3-ζ chain targeted antibody in Jurkat cells treated with CD3/CD28 antibodies with or without FSS (N=3). FIG. 1E shows ZAP70 phosphorylation of Jurkat cells treated with a constant concentration of CD3/CD28 antibodies for 1 h as a function of FSS magnitude (N=3). FIG. 1F shows ZAP70 phosphorylation of Jurkat cells treated with a constant concentration of CD3/CD28 antibodies and a constant magnitude of FSS as a function of time (N=3). Unpaired t tests were used to measure statistical significance between treatment groups. * p<0.05, ** p<0.01, *** p<0.005. Error bars are SD. Linear regression was used to measure correlation and determine if function slopes differed significantly from zero in FIGS. 1E and 1F.

FIGS. 2A to 2F show FSS increases later-stage markers of T cell activation in Jurkat cells. FIG. 2A contains representative images of confocal images of Jurkat cells treated with combinations of FSS and CD3/CD28 antibodies. Area of colocalization between NFAT and DAPI was used to measure NFAT activation (untreated sample n=81, antibody-only sample n=81, antibody-FSS sample n=86, N=3, scale bars=30 μm). Images were acquired on Zeiss 800 LSM with a 40×/1.1 NA water immersion objective. FIG. 2B is a representative flow plot and graph of NF-κB phosphorylation at serine 529 of Jurkat cells treated 1 h with antibodies and FSS (N=3). FIG. 2C is a representative flow plot and graph of cFOS phosphorylation at serine 32 of Jurkat cells treated with antibodies and FSS as a measure of AP-1 activation after 1 h of treatment (N=3). FIG. 2D contains flow plots and graph of TNF-α, IL-2, and IFN-γ expression 24 h after FSS stimulation and antibody treatment (N=3). FIG. 2E contains representative flow plot and graph of CD69 expression 24 h after FSS and antibody treatment (N=3). FIG. 2F contains representative flow plot and graph of CD25 expression 24 h after FSS and antibody treatment (N=3). Unpaired t tests were used to measure statistical significance between treatment groups. * p<0.05, ** p<0.01, *** p<0.005, **** p<0.001. Error bars are SD.

FIGS. 3A to 3F show calcium influx is essential for enhanced T cell activation by FSS in Jurkat cells. FIG. 3A shows ZAP70 phosphorylation of Jurkat cells treated with FSS and antibodies in calcium-free, or calcium-containing buffer (N=3). FIG. 3B shows ZAP70 phosphorylation of Jurkat cells treated with FSS and antibodies with or without the calcium chelator EGTA (N=3). FIG. 3C shows ZAP70 phosphorylation of Jurkat cells treated with FSS and antibodies with or without the mechanosensitive ion channel blocker GsMTx-4 (N=3). FIG. 3D shows ZAP70 phosphorylation of Cas9 control Jurkat cells and Piezo1 KO Jurkat cells treated with FSS and antibodies (N=3). FIG. 3E shows ZAP70 phosphorylation of Jurkat cells treated with FSS and antibodies pretreated with 10 μM CCD for 30 min (N=3). FIG. 3F shows NF-κB phosphorylation of Jurkat cells treated with FSS and antibodies pretreated with 5 μM CSA for 30 min (N=3). Unpaired t tests were used to measure statistical significance between treatment groups. * p<0.05, ** p<0.01, *** p<0.005, **** p<0.001. Error bars are SD.

FIGS. 4A to 4C show FSS enhances activation of primary human T cells. FIG. 4A is a representative flow plot of ZAP70 phosphorylation of CD4-positive T cells. Graphs of ZAP70 phosphorylation for CD4- and CD8-positive T cells treated with FSS and antibodies (N=3). FIG. 4B is a representative flow plot of NF-κB phosphorylation of CD4-positive T cells. Graphs of NF-κB phosphorylation for CD4- and CD8-positive T cells treated with FSS and antibodies (N=3). FIG. 4C is a representative flow plot of IL-2 expression in CD4-positive T cells. Graphs of TNF-α, IL-2, and IFN-γ for CD4- and CD8-positive T cells treated with FSS and antibodies (N=3). Paired t tests were used to measure statistical significance between treatment groups. * p<0.05, ** p<0.01 *** p<0.005. Error bars are SD.

FIG. 5 is a schematic of enhanced T cell activation by FSS and CD3/CD28 antibodies. FSS activates Piezo1, enabling calcium influx. The calcium influx increases ZAP70 phosphorylation, and activation of the transcription factors NFAT, NF-κB, and AP-1. This in turn leads to increased expression of cytokines such as IL-2.

FIG. 6 is a schematic of flow cytometry gating of isolated T cells from peripheral blood.

FIG. 7 contains Annexin V-propidium iodide flow plots of Jurkat cells treated with or without FSS for 1 h. Average cell viability of Jurkat cells treated with or without FSS (N=3). Error bars are SD.

FIG. 8 is a Western blot of Piezo1 and GAPDH expression in Jurkat cells treated with Cas9 and sgRNA targeted to Piezo1, or Jurkat cells treated with Cas9 only (N=1).

FIG. 9 shows shear stress enhances NF-κB phosphorylation in DCs after 1 hour. A cone and plate viscometer was used for 1 h to apply a fluid shear stress to DCs. Cells were placed on a rotator for static conditions, and 10 ug/mL lipopolysaccharide (LPS) was used as a potent stimulator. In these flow plots, a clear shift in NF-κB phosphorylation was observed for the shear conditions.

FIG. 10 shows shear stress enhances NF-κB phosphorylation in DCs after 1 hour. The shift NF-κB phosphorylation can be observed in the histogram. The summary data shows the significance in change in NF-κB phosphorylation for the shear conditions. Interestingly, there was not a significance between shear and shear LPS. ***p<0.005, ****p<0.001.

FIG. 11 shows shear stress enhances AP-1 expression in DCs after 1 hour. A cone and plate viscometer was used for 1 h to apply a fluid shear stress to DCs, with static controls and LPS as a potent stimulator. A shift in AP-1 expression was observed for the shear conditions, although to a lesser extent than for NF-κB.

FIG. 12 shows a significant increase in AP-1 expression for shear conditions, although there was not a significant difference between shear conditions. *p<0.05, **p<0.01.

FIG. 13 shows shear stress shows trend in MHC I expression in DCs after 1 hour. Following 1 hour activation with fluid shear stress, cells were plated for 24 h and analyzed for expression of MHCs.

FIG. 14 shows expression of costimulatory molecules is only enhanced in the presence of LPS. Costimulatory molecule expression is enhanced in the presence of LPS, regardless of whether or not shear stress is present. This trend is observed for CD80, and significant for CD40. The results signify that, if enhanced costimulatory molecule expression is desired, a stimulator is necessary in addition to the fluid shear stress treatments. *p<0.05, **p<0.01, ****p<0.001.

FIG. 15 shows shear stress enhances DC cytokine release after 24 hours. Following 1 hour activation with fluid shear stress, cells were plated for 24 h and analyzed for expression of cytokines and chemokines. There was a significant increase in release of IL-6, IL-12 and CCR7 from DCs that experienced activation via shear stress. There was not a significant difference between shear and shear with LPS. *p<0.05, **p<0.01, ***p<0.005, ****p<0.001.

FIG. 16 shows cytokine release reduced when NF-κB inhibited by caffeic acid phenethyl ester (CAPE). The effect of NF-κB on cytokine release was investigated using caffeic acid (CAPE), which inhibits NF-κB phosphorylation by preventing the translocation of the p65 subunit of NF-κB to the nucleus. It was first shown that CAPE successfully inhibits NF-κB phosphorylation compared to the untreated group. Cytokine release after CAPE treatment was then investigated, a significant reduction in IL-12 release was observed, as well as a clear trend in IL-6 and CCR7 release. **p<0.01, ****p<0.001.

FIG. 17 illustrates a procedure for evaluating Jurkat T cell activation over time following FSS and CD3/CD28 antibody coated bead combination treatment.

FIGS. 18A and 18B show representative flow plots and a graph of enhanced NF-kB phosphorylation in Jurkat cells over time via FSS in the presence and absence of CD3/CD28 antibody coated beads. The shift in NF-kB phosphorylation can be observed in the histogram. The summary graph shows a significant increase in NF-kB phosphorylation in FSS conditions compared to the static conditions and no significant difference between the FSS and FSS with CD3/CD28 antibody conditions from 0 hr-5 days following activation. A two-way ANOVA test was used to measure statistical significance between treatment groups. ***p<0.001, ****p<0.0001. Error bars are SD.

FIGS. 19A and 19B show representative flow plots and a summary graph of enhanced expression of activation marker CD69 in Jurkat cells over time via FSS in the presence and absence of CD3/CD28 antibodies. Error bars are SD for treatment groups>N=2).

FIG. 20 shows a procedure for CAR T cell production and ex vivo activation via FSS and CD3/CD28 combination treatment. 24 hours following ex vivo activation of T cells, lentiviral particles are added to T-cell culture media, which encode a prostate-specific membrane antigen (PSMA) targeting CAR construct. The protocol can be used for ex vivo activation of T cells prior to the transduction of any antigen targeting CAR construct.

FIGS. 21A and 21B show enhanced CAR T cell transduction with FSS and CD3/CD8 antibody treatment, in Jurkat cells and primary prostate cancer patient T cells, respectively. To obtain primary T cells, leukocytes were isolated from a whole blood sample using Ficoll™ separation and T cells were magnetically separated via negative selection. There is an observed increase in transduction efficiency, normalized to the control T cells, when evaluating the Median Fluorescence Intensity (MFI) of PSMA expression (FITC) in flow cytometry. The shift in PSMA expression can be observed in the histograms. (N=1).

FIG. 22 a procedure for co-culture of activated primary CAR T cells via FSS and CD3/CD28 antibody combination treatment with PC3 prostate cancer cells. A representative forward-scatter vs. side-scatter flow cytometry plot is shown of the T cell and PC3 cell populations. Moreover, PC3 cells were labeled with a cell tracking dye prior to the addition of T cells to further delineate the cell populations, enabling the downstream analysis of only PC3 cells.

FIG. 23 shows that the cytotoxicity of primary non-CAR T cells is unaffected by activation in PC3 prostate cancer cells. To obtain primary T cells, leukocytes were isolated from a whole blood sample using Ficoll™ separation and T cells were magnetically separated via negative selection. A two-way ANOVA test was used to measure statistical significance between treatment groups (ns, p>0.05). Error bars are SD. (N=3)

FIG. 24 shows that the cytotoxicity of primary prostate cancer patient CAR T cells is enhanced following ex vivo FSS and CD3/CD8 activation. CAR T cells activated by fluid shear stress exhibit a decrease in PC3 cell viability. To obtain primary T cells, leukocytes were isolated from a whole blood sample using Ficoll™ separation and T cells were magnetically separated via negative selection. (N=1).

FIGS. 25A to 25E show shear stress enhances NF-κB phosphorylation in DCs at low shear times and rates.

FIGS. 26A and 26B show an updated mechanism of FSS activation of DCs, including the effect that Piezo1 inhibitor, GsMTx4, has on NF-κB phosphorylation.

FIG. 27 shows a procedure for evaluating the effects of pre-coating with BSA. Cone-and-plate viscometers are pre-coated in 5% bovine serum albumin (BSA) for blocking. It is then verified that this is not causing differences between static and shear values.

FIGS. 28A and 28B show blocking with BSA does not significantly enhance AP-1 expression compared to FSS.

FIGS. 29A to 29C show increased cytokine release following FSS. Experiments were performed using R&D Systems Proteome Profiler Cytokine Array Kit, Panel A.

FIG. 30 shows increased cytokine release following FSS. Significant increase in cytokine release was observed for the FSS group compared to both the static and BSA controls for TNF-α, sICAM-1, CCL3, CXCL2, and CCL5. Trends were observed for other key cytokines. *p<0.05, **p<0.01, ***p<0.005, ****p<0.001. Error bars are in SE.

FIG. 31 shows similar cytokine profiles observed after mouse serum albumin (MSA)-pre-coating.

FIG. 32 shows procedure for measuring glucose uptake using 2-[N-(7-nitrobenz-2-oxa-1,3-diazol-4-yl) amino]-2-deoxy-d-glucose (2-NBDG). Fluorescent tracer used to monitor glucose uptake into cells. 2-NBDG is a derivative of glucose modified with an amino group.

FIG. 33 shows trend in enhanced glucose uptake following FSS, measured using 2-NBDG.

FIG. 34 shows procedure for bone marrow dendritic cell (BMDC) isolation.

FIGS. 35A and 35B show Phospho-proteins and costimulatory molecule expression enhanced in BMDCs with FSS.

FIG. 36 shows procedure for DC isolation and analysis of prostate cancer patient blood samples.

FIG. 37 shows population analysis and NF-κB phosphorylation for DCs isolated from prostate cancer patients.

FIG. 38 shows AP-1, costimulatory molecule, and cytokine expression for DCs isolated from prostate cancer patients.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, engineering, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Immune Effector Cells

Disclosed herein are immune effector cells for use in immunotherapy that have been activated ex vivo using the disclosed methods. The immune effector cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cell populations can be accomplished by negative selection using a combination of antibodies directed at surface markers unique to the negatively selected cells.

In some embodiments, the immune effector cells comprise any leukocyte involved in defending the body against infectious disease and foreign materials. For example, the immune effector cells can comprise lymphocytes, dendritic cells, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (Tc cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

Dendritic cells (DCs) are antigen-presenting cells (also known as accessory cells) of the mammalian immune system. Their main function is to process antigen material and present it on the cell surface to the T cells of the immune system. They act as messengers between the innate and the adaptive immune systems. The most common division of dendritic cells is “myeloid” vs. “plasmacytoid dendritic cell” (lymphoid). The markers BDCA-2, BDCA-3, and BDCA-4 can be used to discriminate among the types. Lymphoid and myeloid DCs evolve from lymphoid and myeloid precursors, respectively, and thus are of hematopoietic origin. By contrast, follicular dendritic cells (FDC) are probably of mesenchymal rather than hematopoietic origin and do not express MHC class II, but are so named because they are located in lymphoid follicles and have long “dendritic” processes.

In some embodiments, the T cells comprise a mixture of CD4+ cells. In other embodiments, the T cells are enriched for one or more subsets based on cell surface expression. For example, in some cases, the T cells comprise cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-1-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

In some embodiments, the immune effector cells are engineered to express chimeric receptors, such as CAR polypeptides (also referred to herein as “CAR-T cells”). CARs are generally made up of three domains: an ectodomain, a transmembrane domain, and an endodomain. The ectodomain comprises the EGFR or MUC1-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally one or multiple co-stimulatory signaling regions (CSR).

### Activation and Expansion

As disclosed herein, fluid shear stress (FSS) enhances immune effector cell activation. Therefore, disclosed herein is a method for activating immune effector cells ex vivo that involves applying an effective amount of fluid shear stress to the cells.

The shear stress can be applied using any suitable flow device which is capable of inducing flow of the fluid medium. For example, the flow device can be a cone-and-plate device or a parallel plate flow device. In some embodiments, the device is a closed loop peristaltic pump system, electrokinetically-driven microfluidic flow, syringe pump flow, or gravity-driven flow on an oscillating orbitor or tumbler device.

The shear stress applied upon the immune effector cell can be about 0.1 dynes/cm² to about 20 dynes/cm². For example, the shear stress applied upon at least one tumor cell type can be about 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, or 20 dynes/cm². In some embodiments, the cells are exposed to shear stresses of about of 0.5-20 dyne/cm², about 0.5-15 dyne/cm², about 0.5-10 dyne/cm², about 1-20 dyne/cm², about 1-15 dyne/cm², or about 1-10 dyne/cm².

In some embodiments, the cells are exposed to the shear stress for between about 5 minutes to about 6 hours, such as about 15 minutes to about 2 hours, including about 5, 6, 7, 8, 9, 10, 11, 12, 13, 14, 15, 16, 17, 18, 19, 20, 25, 30, 35, 40, 45, 50, 60 minutes, and including about 1, 2, 3, 4, 5, or 6 hours.

The shear stress can be applied with a shear rate of about 1-20,000 1/s.

In some embodiments, the viscosity of the media is adjusted, in order to provide the proper shear force. For example, in some embodiments, additives such as glycerol, pluronic F68, dextran, polyethylene glycol (PEG), are used enhance the viscosity of the medium.

In some embodiments, the appropriate shear stress is determined based on Piezo1 expression by the immune effector cells. Therefore, in some embodiments, the method further involves assaying the immune effector cells for Piezo1 expression, and selecting a fluid shear stress, viscosity, time, or any combination thereof based on the Piezo1 expression levels.

Shear stress can be measured, for example, with a MicroS3.v10 probe (Viosense Corporation, Pasadena, Calif). The MicroS3 probe uses optical Doppler velocimetry to measure shear stress within 166 μm of its surface. Shear stress=f_{Doppler}·K·μ where f_{Doppler} is the mean frequency (Hz) of the Doppler shift in the area sampled by the sensor and is calculated by Fast-Fourier Transformation; K is the fringe divergence, a constant characterized for each sensor; and p is the dynamic viscosity and is equal to the product of the kinematic viscosity (ν) and the density (ρ). The Reynolds' number can be calculated as ω R²/ν where ω is the rotational speed of the orbital shaker, R is the radius of rotation of the orbital shaker (0.975 mm), and u is the kinematic viscosity (1.012×10⁻⁶ m²/s). Shear stress can also be measured using micro particle image velocimetry (μPIV), or predicted using computational fluid dynamics (CFD) calculation of the flow based on the device geometry and the bulk flow rate settings.

The flow device can be a cone-and-plate device substantially as described in U.S. Pat. No. 7,811,782, the contents of each of which are hereby incorporated by reference with respect to their teachings regarding cone-and-plate flow devices.

For laminar shear flow in a tube, the shear stresses are greatest near the tube wall and are zero in the center of the tube where fluid is simply translating downstream. Wall shear stress t, and transit time tₜᵣₐₙₛ it may be defined as: t_{w} (dyne/cm²)=4 m Q/(πR³) and tₜᵣₐₙₛᵢₜ=(L A)/Q for volumetric flow rate Q though a tube of cross-sectional area A=πR² where: Q=v_{avg} A for Q[=]cm³/s, v_{avg}[=]cm/s, and A[=]cm². The average transit time across a length of tubing L is defined from v_{avg}=L/tₜᵣₐₙₛᵢₜ such that tₜᵣₐₙₛᵢₜ=L/v_{avg} L A/Q. The viscosity of water is 0.01 Poise at room temperature. At constant flow rate and geometry, increasing the viscosity will increase the shear forces. Entrance length effects are fairly minimal in small diameter tubes.

In some embodiments, the immune effector cells are further exposed to one or more activating agents before, during, or after shear stress application. For example, the activating agent can be present in the fluid media.

Immune effector cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle:cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 104 to 109 T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Activation of dendritic cells (DCs) has been shown to occur in response to a number of different stimuli, including signals derived from dead or damaged cells or from infection. For example, DCs can be activated by exposure to whole pathogens, components of microorganisms (e.g., LPS, dsRNA, CpG DNA, toxins), and cytokines induced by infection, such as type I IFN (IFN-αβ) and IL-15.

Various protocols have been used to isolate and preferentially expand primary NK cells from PBMC. The common principle is a combination of cell selection and depletion using immunomagnetic beads. These protocols use leukapheresis products for the clinical-grade purification of NK cells by depleting CD3 cells followed by selection of CD56 cells or in combination with subsequent short-term (14-day) expansion with IL-2. Clinical-grade expansion of NK cells in lymphokine-activated killer (LAK) cell cultures for 28 days with IL-15 has been reported. NK cell expansion requires multiple signals for survival, proliferation and activation. Thus, expansion strategies have been focused either to substitute these factors using autologous feeder cells and/or to use genetically modified allogeneic feeder cells. Functional activity is defined by cytotoxicity against various malignant cell lines and expression pattern of NK cell receptor (cluster of differentiation (CD)-16, natural killer group-2 member D (NKG2D), CD69, NKp30, NKp44, NKp46 and CD158b. Expansion of NK with autologous PBMC as feeder cells has been shown to generate functional active NK cells with a therapeutic cell dosage. Using GMP-compliant components and autologous feeder cells, purified NK cells were effectively expanded (2500-fold at day 17). Similarly, large-scale expansion of GMP-compliant NK cells with cytolytic activity against tumor cells can be produced using autologous PBMCs in the presence of OKT3 and IL-2 at 14 days. Other feeder cells such as Jurkat T-lymphoblast subline KL-1, the leukemia cell line K562, genetically altered to express membrane-bound form of IL-15 and the 4-1BB (CD137L), or engineered to express membrane-bound IL-21 along with CD137L, can be used to expand NK cells. In some embodiments, autologous NK cells can be activated and potentiated through systemic administration of cytokines like interleukin (IL)-2, IL-12, IL-15, IL-18, IL-21 and type I IFNs.

### Therapeutic Methods

Immune effector cells activated by the disclosed methods can be used to treat cancers. Briefly, pharmaceutical compositions may comprise a target cell population as described herein, in combination with one or more pharmaceutically or physiologically acceptable carriers, diluents or excipients. Such compositions may comprise buffers such as neutral-buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. The quantity and frequency of administration will be determined by such factors as the condition of the patient, and the severity of the patient's disease, although appropriate dosages may be determined by clinical trials.

When a “therapeutic amount” is indicated, the precise amount of the compositions of the present invention to be administered can be determined by a physician with consideration of individual differences in age, weight, extent of transplantation, and condition of the patient (subject). It can generally be stated that a pharmaceutical composition comprising the immune effector cells described herein may be administered at a dosage of 10⁴ to 10⁹ cells/kg body weight, such as 10⁵ to 10⁶ cells/kg body weight, including all integer values within those ranges. Immune effector cells compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the disclosed compositions may be carried out in any convenient manner, including by injection, transfusion, or implantation. The compositions described herein may be administered to a patient subcutaneously, intradermally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a site of transplantation.

In certain embodiments, the disclosed immune effector cells are administered to a patient in conjunction with (e.g., before, simultaneously or following) any number of relevant treatment modalities, including but not limited to thalidomide, dexamethasone, bortezomib, and lenalidomide. In further embodiments, the immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

One primary concern with immune effector cells as a form of “living therapeutic” is their manipulability in vivo and their potential immune-stimulating side effects. To better control immune effector cell therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promote elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a first antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tissue, and is advantageous in situations where certain normal tissues may express very low levels of an antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason, these too are also referred to as a “split CAR”.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Fluid Shear Stress Enhances T Cell Activation Through Piezo1

Exposing T cells ex vivo to mechanical forces may be another method of boosting T cell activation and production. In vivo, optimal T cell activation is both a mechanical and biochemical process (Lim, T. S. et al. J. Immunol. 2011 187:258-265; Blumenthal, D. et al. eLife. 2020 9:e55995). For example, the knockdown of the mechanotransductive protein Piezo1 was recently shown to reduce the expansion of T cells activated by mouse dendritic cells (Liu, C. S. C. et al. J. Immunol. 2018 200:1255-1260). Piezo1 is a mechanosensitive ion channel that opens in response to physical forces, such as fluid shear stress (FSS), and allows for calcium influx (Syeda, R. et al. Cell Rep. 2016 17:1739-1746; Hope, J. M. et al. Cell Death Dis. 2019 10:1-15). Calcium influx causes Piezo1 to transduce physical stimuli into biochemical responses, since calcium is a second messenger involved in multiple signaling pathways. One of these pathways is T cell activation, as calcium influx increases the activation of the transcription factors NFAT, NF-κB, and AP-1 (Borgne, M. L. et al. J. Immunol. 2016 196:1471-1479; Liu, et X. al. J. Biol. Chem. 2016 291:8440-8452; Feske, S. et al. Nat. Immunol. 2001 2:316-324; Joseph, N. et al. Biochim Biophys Acta 2014 1838(2):557-568; Fehr, T. et al. Blood. 2010 115:1280-1287). These transcription factors then induce the production of cytokines important in sustained T cell activation, differentiation, and cytotoxicity (Vaeth, M. et al. F1000Research. 2018 7; Peng, S. L. et al. Immunity. 2001 14:13-20). Fluid shear stress (FSS) induces calcium influx in single T cells through the use of a micropipette apparatus (Li, Y.-C. et al. J. Immunol. 2010 184:5959-5963), suggesting that that FSS may be leveraged to activate Piezo1 ex vivo. The activation of Piezo1 would then lead to increased activation of T cells and cytokine secretion.

Treating T cells with FSS in this controlled setting also allows for the investigation of the role of FSS in T cell activation in pathophysiological contexts. Hypertension is a disease in which blood flow is altered, is associated with abnormal cytokine levels, and is correlated with multiple autoimmune disorders (Mirhafez, S. R. et al. J. Am. Soc. Hypertens. JASH. 2014 8:614-623; Taylor, E. B. et al. Br. J. Pharmacol. 2019 176:1897-1913). In certain regions of the vascular network of hypertensive patients, blood flow velocity is reduced, reducing FSS. In other areas, blood flow velocity is increased, raising the FSS magnitude (Perret, R. S. et al. Ultrasound Med. Biol. 2000 26:1387-1391).

This study shows that FSS enhances T cell activation. This was investigated by treating T cells ex vivo with FSS using a cone-and-plate viscometer. It was found that short FSS treatments in combination with exposure to soluble CD3/CD28 antibodies enhanced the activation of important signaling proteins in T cell activation, and boosted the expression of cytokines important in sustained T cell function. The enhanced activation of T cells was found to rely on calcium and activation of the mechanosensitive ion channel Piezol. This suggests that FSS may have the potential to improve ex vivo T cell activation for adoptive therapies. Additionally, this observation suggests that Piezol and other mechanosensitive ion channels may be therapeutic targets for autoimmune diseases.

### Results

FSS Enhances ZAP70 Phosphorylation in Jurkat Cells Treated with CD3/CD28 Antibodies.

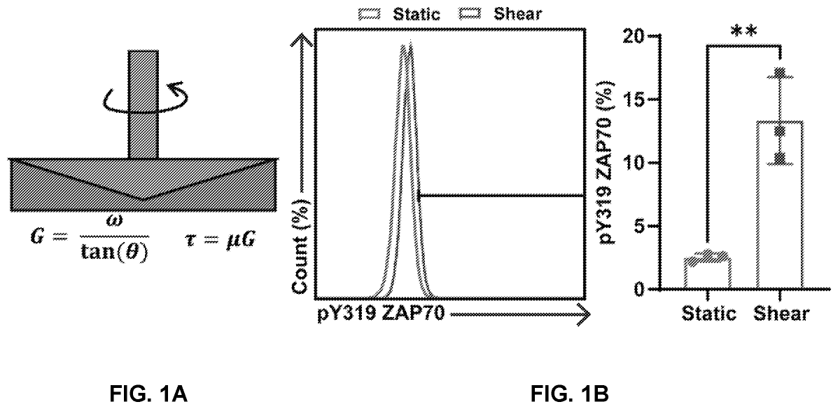

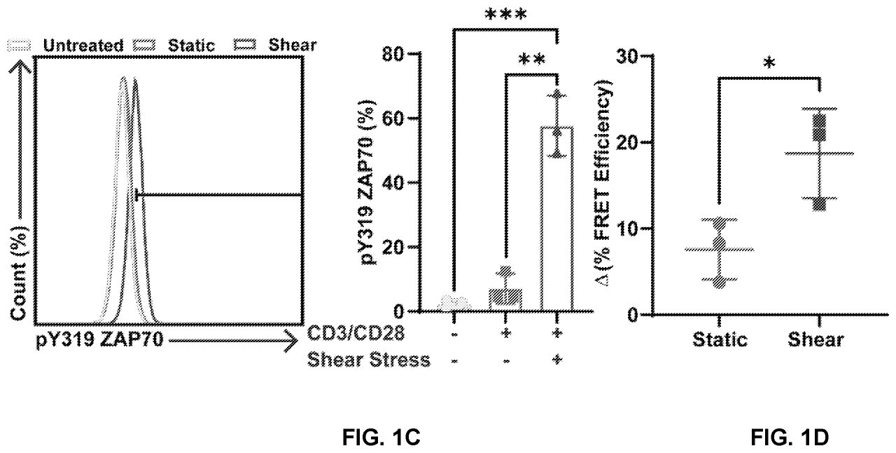

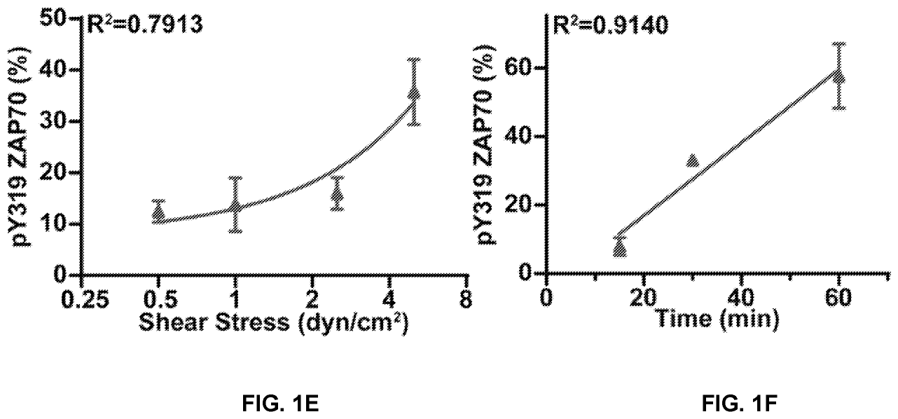

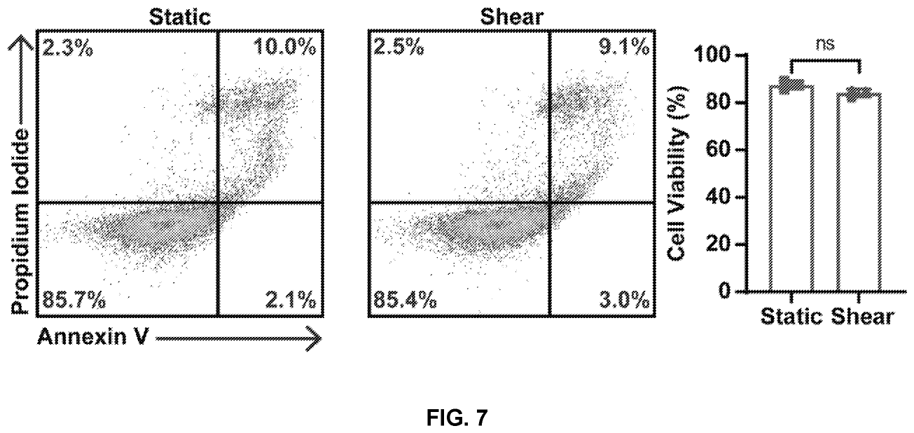

The immortalized Jurkat cell line was used to model T cell activation by CD3/CD28 antibodies in combination with FSS in vitro. Jurkat cells were incubated with soluble CD3/CD28 antibodies with or without FSS for 1 h. Cone-and-plate viscometers were used to expose the cells to 5.0 dyn/cm² FSS (FIG. 1A). However, FSS at sufficient magnitude has previously been shown to be cytotoxic to cells (Hope, J. M. et al. J. Cell Sci. 2021 134). To determine if this level of FSS exposure was cytotoxic to the cells, an annexin V assay was done to measure cell viability. 1 h of FSS treatment at 5.0 dyn/cm² did not significantly reduce cell viability (FIG. 7). Jurkat cells were first treated with FSS without antibodies to determine the activating effect of FSS alone. This was quantified by measuring ZAP70 phosphorylation, which is phosphorylated following the activation of CD3 and is a necessary step for T cell activation (Torchia, M. L. G. et al. Proc. Natl. Acad. Sci. 2018 115:2174-2179). FSS alone caused only a 10% increase in ZAP70 phosphorylation compared to the static control (FIG. 1B). When CD3/CD28 antibodies were combined with the FSS treatment, ZAP70 phosphorylation was significantly and dramatically increased in comparison to antibody only or the untreated controls. The increase in ZAP70 phosphorylation was about 50% greater when FSS was used with antibodies, compared to the ZAP70 phosphorylation of T cells treated with antibodies alone (FIG. 1C). FRET efficiency was also used as a measure of ZAP70 activation in Jurkat cells, as ZAP70 is recruited to the ζ-chain of CD3 upon T cell activation (Lysechko, T. L. et al. J. Immunol. 2005 174:7807-7814). When FSS was used in combination with CD3/CD28 antibodies, a significant increase in FRET efficiency was observed compared to the static CD3/CD28 antibody control, suggesting that FSS increases the recruitment of ZAP70 to the ζ-chain of CD3 (FIG. 1D). Jurkat cells were then treated with varying amounts of FSS to determine the “dose response” of T cell activation to shear stress. A positive correlation of increasing ZAP70 phosphorylation with increasing amounts of FSS was observed, with R²=0.7913. Additionally, the slope of the best fit line was significantly different from zero (FIG. 1G). The dependence of Jurkat ZAP70 phosphorylation on FSS-antibody treatment time was also measured. There was a significant increase in ZAP70 phosphorylation with increasing FSS treatment times, with R²=0.9140. The best fit line between treatment time and ZAP70 phosphorylation was significantly different from zero (FIG. 1F).

FSS in Combination with CD3/CD28 Antibodies Increases the Expression of Later Stage T Cell Activation Markers in Jurkat Cells.

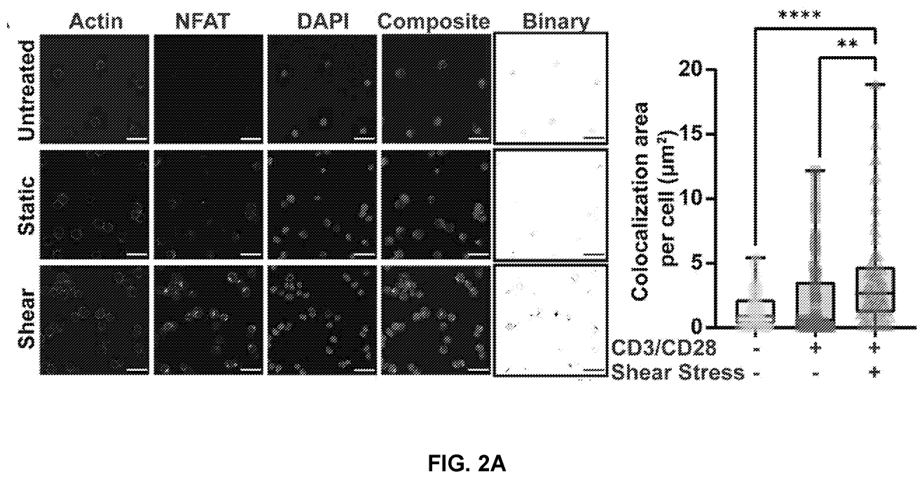

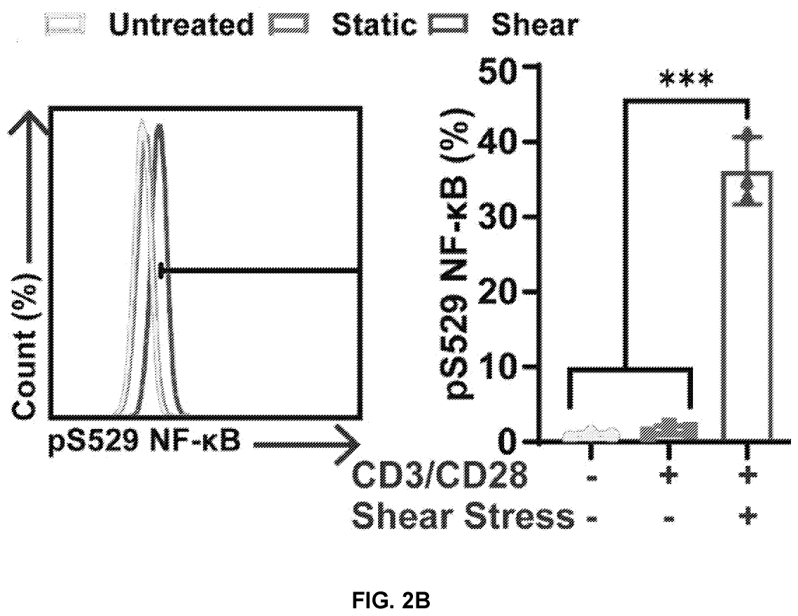

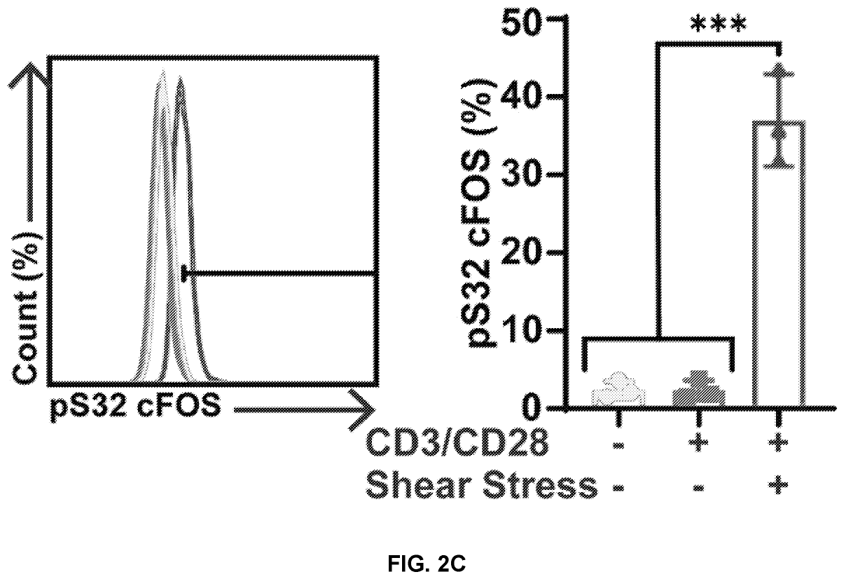

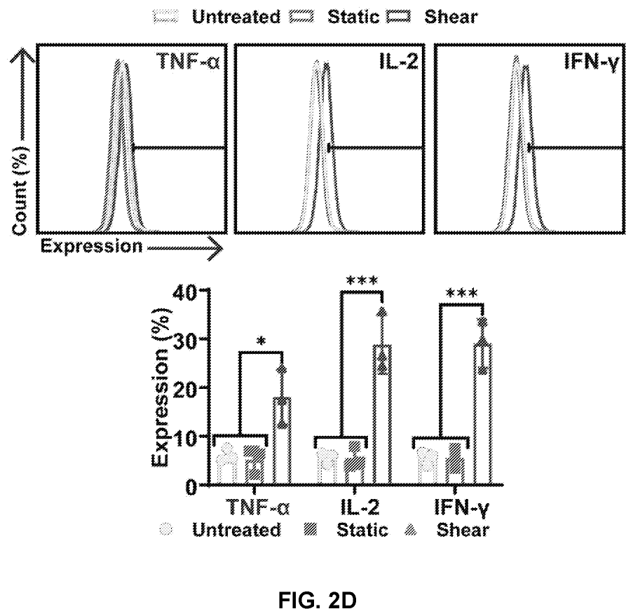

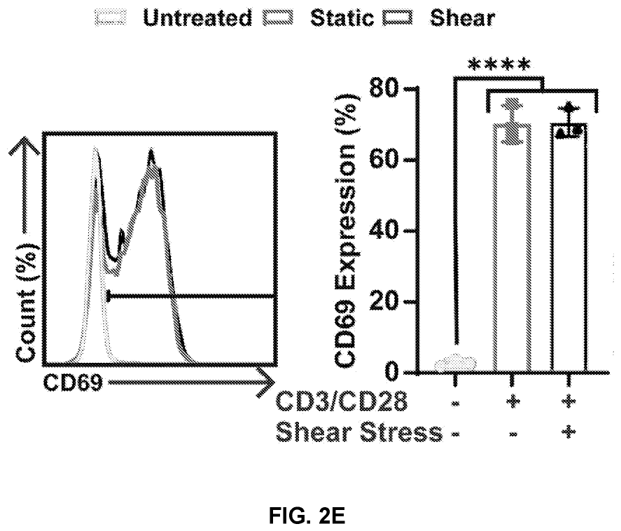

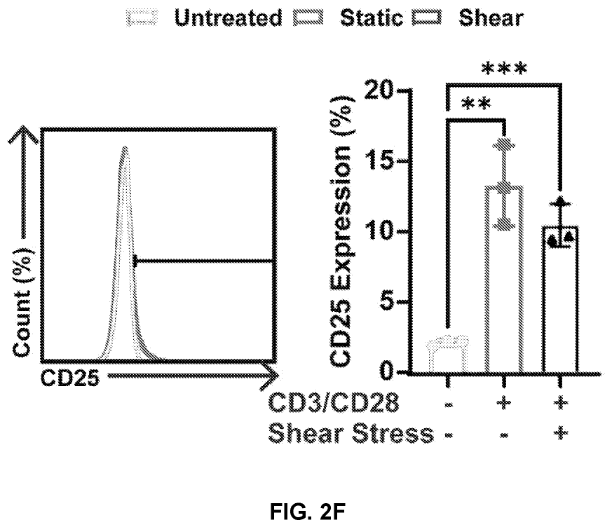

For proper T cell activation, the transcription factors NFAT, NF-κB, and AP-1 need to be activated for the transcription of important proteins and cytokines, such as TNF-α, IL-2, and IFN-γ (Naito, T. et al. Int. Immunol. 2011 23:661-668). The activation of each transcription factor was measured after 1 h of stimulation with FSS and CD3/CD28 antibodies. NFAT activation of Jurkat cells was measured by quantifying the colocalization of NFAT with the nucleus using confocal microscopy, since only the active conformation of NFAT reveals a nuclear localization signal (Gabriel, C. H. et al. J. Biol. Chem. 2016 291:24172-24187). The colocalization was measured via antibody staining for NFAT and DAPI staining of the nucleus. When the Jurkat cells were treated with CD3/CD28 antibodies and FSS, a significant increase in NFAT-nucleus colocalization was observed relative to untreated, or antibody-only treated Jurkat cells (FIG. 2A). NF-κB activation was quantified by measuring the phosphorylation of NF-κB using flow cytometry, as NF-κB phosphorylation is indicative of its activation (Christian, F. et al. Cells. 2016 5). AP-1 activation was quantified by measuring the phosphorylation of cFOS, since cFOS must be phosphorylated to form the AP-1 complex (Tanos, T. et al. J. Biol. Chem. 2005 280:18842-18852). FSS in combination with CD3/CD28 antibodies significantly increased the activation of NF-κB and AP-1 compared to untreated and antibody-only treated Jurkat cells (FIGS. 2B,C).

The expression of the cytokines TNF-α, IL-2, and IFN-γ were measured 24 h after 1 h of FSS treatment, to determine if the increased activation of the three transcription factors correlated with increased expression of proteins necessary for sustained T cell function and activation. The expression of each cytokine was significantly increased when Jurkat cells were treated with FSS for 1 h compared to both control groups (FIG. 2D). The expression of the activation markers CD69 and CD25 were also measured 24 h after FSS treatment. Both the FSS-antibody treated group and the antibody-only treated group showed a significant increase in CD69 and CD25 expression compared to the untreated control. There was no significant difference in CD69 and CD25 expression between either treatment group (FIGS. 2E,F).

### FSS Enhancement of T Cell Activation in Jurkat Cells is Piezo1 and Calcium Dependent.

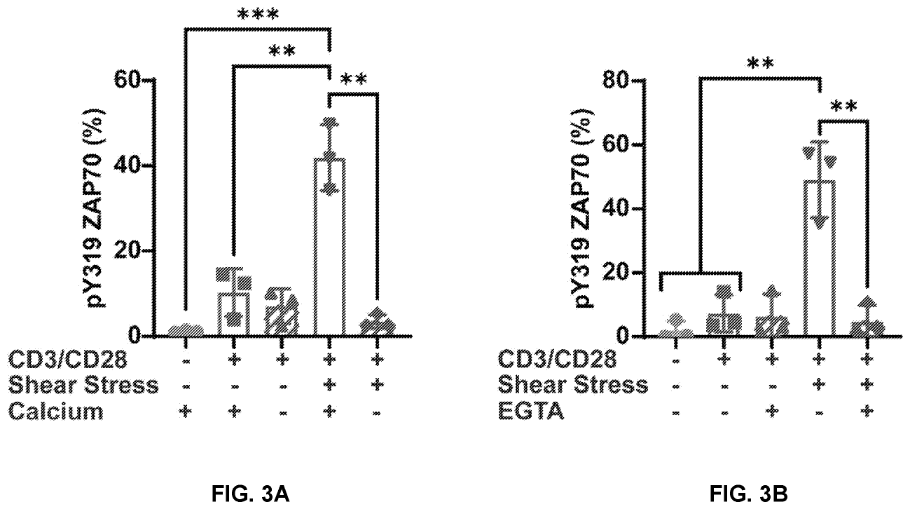

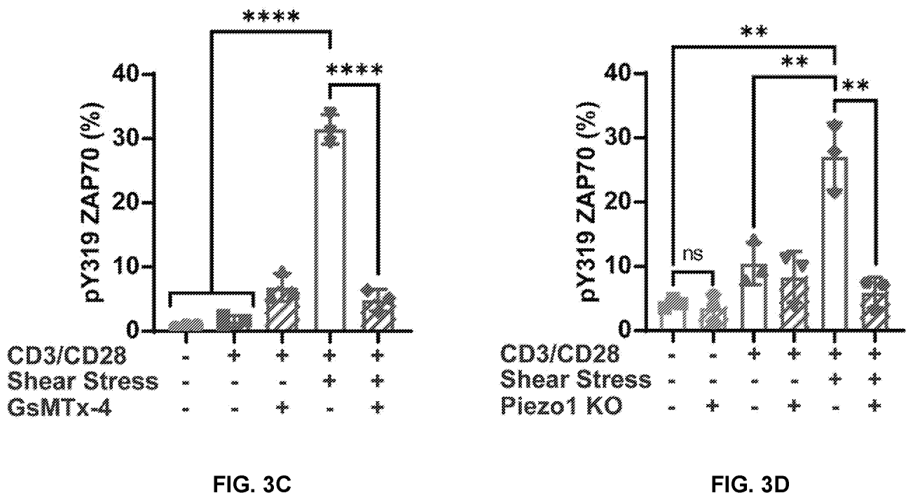

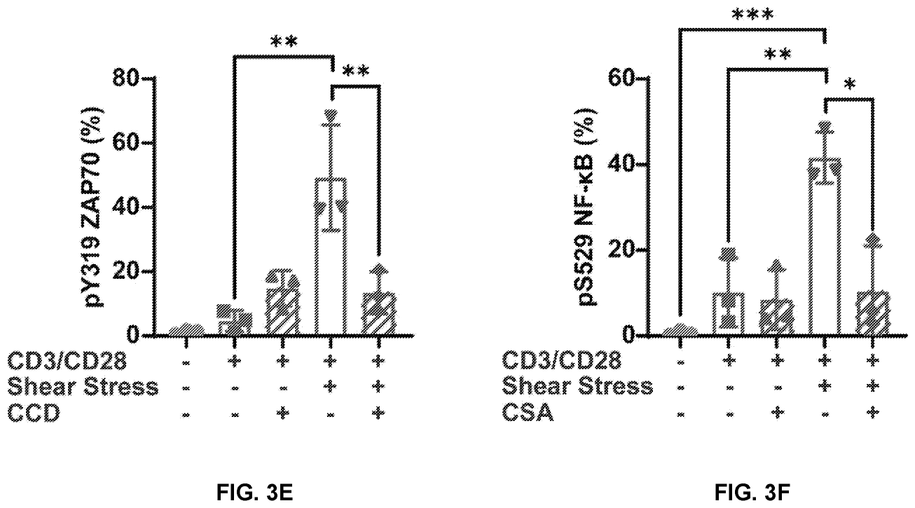

To determine if FSS-enhanced T cell activation acted through calcium influx, Jurkat cells were treated for 1 h with combinations of antibodies plus FSS in calcium-free, or calcium-containing HBSS buffer. Jurkat cells treated with FSS and antibodies in calcium-containing buffer showed a significant increase in ZAP70 phosphorylation compared to Jurkat cells treated with FSS and antibodies in calcium-free buffer. The Jurkat cells treated with FSS and antibodies in calcium-free buffer did not show a significant increase in ZAP70 phosphorylation compared to Jurkat cells treated with or without antibodies in calcium-free buffer (FIG. 3A). Next, Jurkat cells were pretreated with or without the calcium chelator EGTA at a concentration of 2 mM for 30 min. EGTA inhibited the enhanced phosphorylation of ZAP70 that was previously observed in response to FSS and antibody treatment in Jurkat cells. Jurkat cells treated with EGTA, FSS, and antibodies showed a significant reduction in ZAP70 phosphorylation compared to Jurkat cells treated with FSS and antibodies only (FIG. 3B).

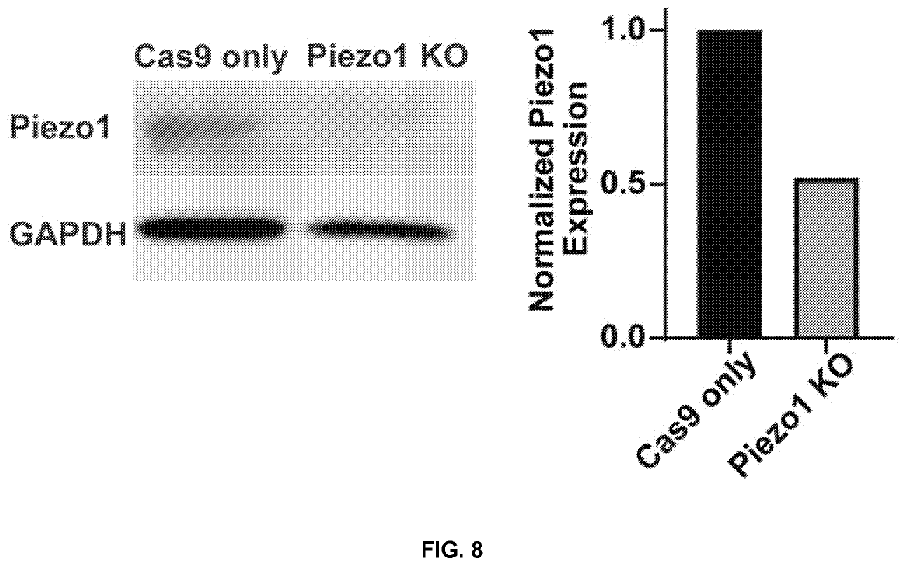

Piezol is a mechanosensitive ion channel that can be activated by FSS and is known to transport calcium when active. To determine if Piezol may play a role in the enhanced T cell activation by FSS, Jurkat cells were treated with 10 μM GsMTx-4 30 min prior to FSS and antibody treatment. GsMTx-4 is a relatively specific inhibitor of Piezol, but is known to inhibit other mechanosensitive channels as well, such as TRPC6 (Bae, C. et al. Biochemistry. 2011 50:6295-6300). GsMTx-4 caused a significant reduction in ZAP70 phosphorylation for Jurkat cells treated with FSS and antibodies in comparison to Jurkat cells treated with FSS, antibodies, and no GsMTx-4 (FIG. 3C). This indicates that FSS enhances T cell activation by inducing the activation of mechanosensitive ion channels that result in calcium influx. To more specifically quantify the degree to which Piezol is the ion channel responsible for the enhanced activation of Jurkat cells by FSS, Piezol was knocked out in Jurkat cells using CRISPR/Cas9 technology. The knockout (KO) of Piezol was confirmed via western blot (FIG. 8). In Jurkat Piezol KO cells, FSS treatment did not increase ZAP70 phosphorylation compared to untreated, or antibody-only treated cells. However, in the Cas9 control Jurkat cells the FSS treatment significantly increased ZAP70 phosphorylation compared to untreated and antibody-only treated cells. Additionally, ZAP70 phosphorylation was significantly greater in Cas9 control Jurkat cells compared to Jurkat Piezol KO cells when both were treated with FSS (FIG. 3D).

Downstream effects of calcium influx were also investigated to identify if they play a role in enhancing T cell activation in the presence of FSS. Actin polymerization has previously been identified as being necessary for efficient T cell activation, and is regulated by calcium influx (Liu, C. S. C. et al. J. Immunol. 2018 200:1255-1260). Jurkat cells were pretreated with 10 μM of CCD 30 min prior to FSS treatment to inhibit actin polymerization. CCD significantly reduced the increased ZAP70 phosphorylation associated with FSS-antibody treatment, while not significantly altering the activation of Jurkat cells under the antibody-only condition (FIG. 3E).

Calcinuerin is a phosphatase that is activated downstream of calcium influx and has previously been associated with NF-κB activation (Palkowitsch, L. et al. J. Biol. Chem. 2011 286:7522-7534). To determine if calcinuerin is activated by calcium influx to increase transcription factor activation, calcineurin was inhibited by pretreating the Jurkat cells with 5 μM CSA for 30 min. CSA treatment significantly reduced NF-κB phosphorylation in Jurkat cells treated with FSS and antibodies for 1 h compared to Jurkat cells treated with FSS without CSA, suggesting that FSS activates calcineurin to boost transcription factor activation (FIG. 3F).

### FSS Enhances Activation of Primary T Cells.

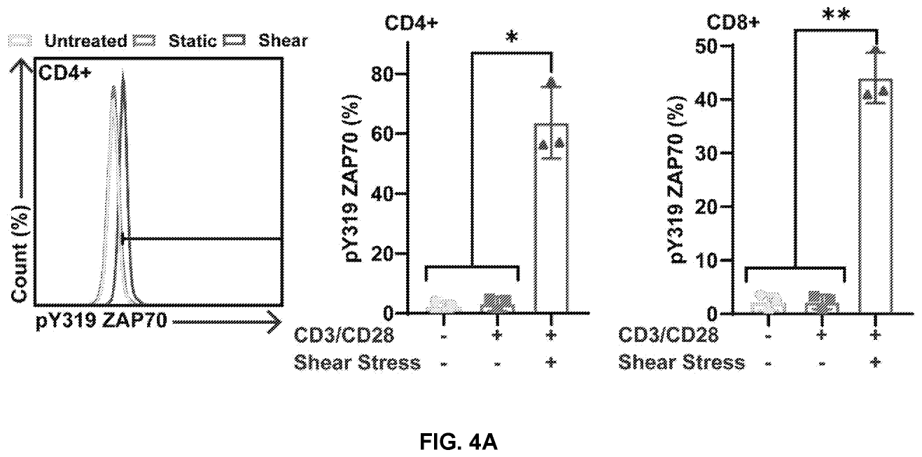

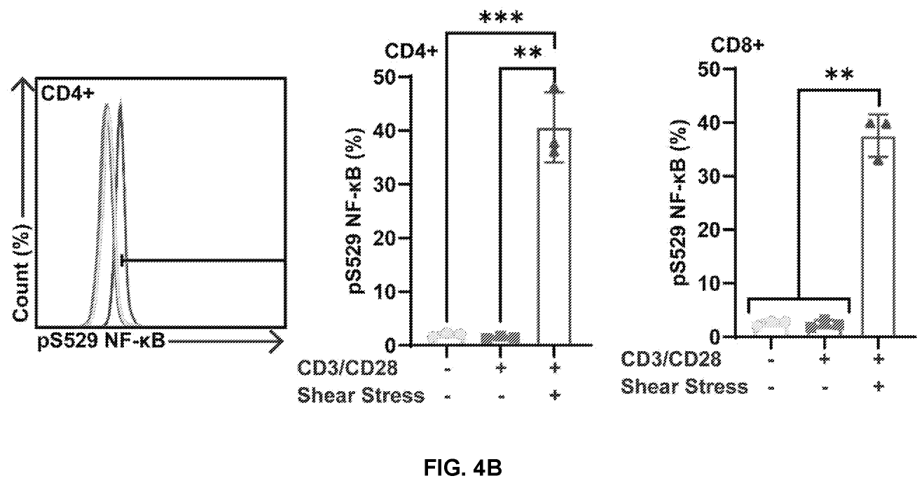

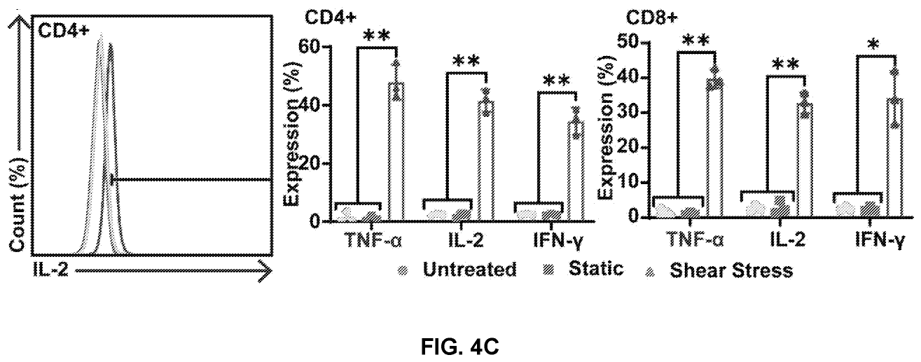

To determine if the activating effects of FSS treatment are relevant to T cell therapies in the clinic, primary human T cells were treated with antibodies and FSS for 1 h. Primary T cells were isolated from the blood of 3 healthy donors using magnetic bead negative selection. Immediately following isolation, the T cells were treated with combinations of FSS and CD3/CD28 antibodies. The cells were stained for CD4 and CD8 to identify the helper and cytotoxic T cell subpopulations, respectively. The cells were also stained for various markers of T cell activation. FSS enhanced the phosphorylation of ZAP70 in both CD4- and CD8-positive T cell subpopulations compared to untreated and antibody-only control groups (FIG. 4A). This trend was also apparent with NF-κB phosphorylation where both the CD4- and CD8-positive T cells showed a significant increase in NF-κB phosphorylation compared to the two control groups (FIG. 4B). As mentioned above, cytokine expression is another essential step for T cell activation and function. Therefore, 24 h after 1 h of FSS treatment, the expression of TNF-α, IL-2, and IFN-γ was measured for primary T cells in both subpopulations. Again, the observations in Jurkat cells were recapitulated ex vivo with both primary T cell subpopulations. In CD4- and CD8-positive T cells there was a significant increase in all three cytokines when the T cells were treated with FSS and CD3/CD28 antibodies relative to untreated and antibody-only controls (FIG. 4C).

### Discussion

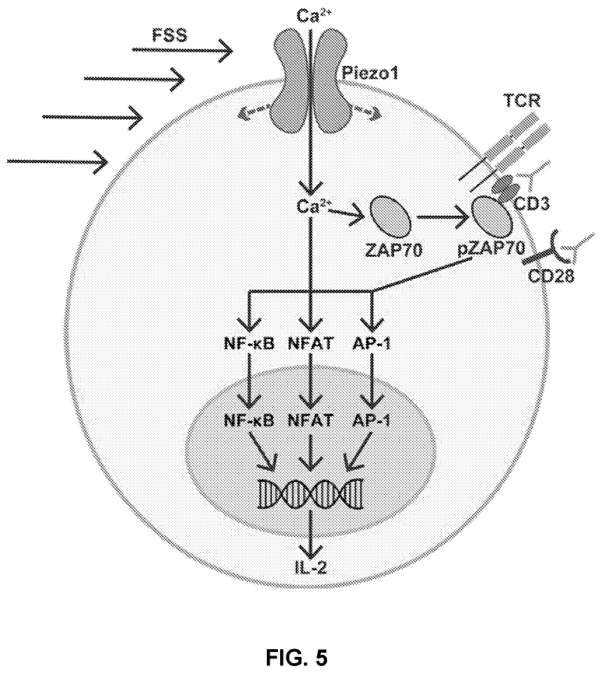

The present study is demonstrated that FSS can enhance T cell activation (FIG. 5). FSS with CD3/CD28 antibodies was found to significantly enhance ZAP70 phosphorylation in immortalized Jurkat cells, and CD4- and CD8-positive human T cells (FIGS. 1,4). FSS was also found to increase the activation of the three major transcription factors associated with T cell activation in Jurkat cells (FIG. 2). These results are consistent with NF-κB in both subsets of primary T cells (FIG. 4). Finally, in both the Jurkat and primary T cells, upregulation of TNF-α, IL-2 and IFN-γ occurred when the cells were treated with FSS and activating antibodies (FIGS. 2,4). These results suggest that FSS, such as that produced in the cone-and-plate viscometer, has the potential to enhance the activation of T cells in future T cell applications.

One of the primary factors that determines if CAR T cell therapy will be effective in a patient is how well the CAR T cells persist after they are administered (Jensen, M. C. et al. Biol Blood Marrow Transplant. 2010 16(9):1245-56). The increased expression of cytokines may be particularly advantageous, as increased cytokine secretion has been shown to improve CAR T cell tumor targeting and persistence (Jin, J. et al. Am. J. Cancer Res. 2020 10:4038-4055; Xue, Q. et al. J. Immunother. Cancer. 2017 5:85). For example, treating cancer patients with exogenous IL-2 in combination with adoptive T cell therapies increases the in vivo persistence and proliferation of the T cells, resulting in better patient outcomes (Rosenberg, S. A. et al. Clin Cancer Res. 2011 17(13):4550-7). Also, Incubating T cells with IL-2 ex vivo is known to boost the proliferation and cancer cytotoxicity of the T cells (Besser, M. J. et al. Cytotherapy. 2009 11:206-217; Levine, B. L. et al. Mol Ther Methods Clin Dev. 2016 4:92-101). Here, FSS increases the expression of IL-2 by activated T cells (FIGS. 2,4). This suggests that activating T cells with FSS prior to their administration may increase T cell persistence due to the increased amounts of IL-2 produced by the FSS-treated T cells. The enhanced IL-2 secretion could boost the proliferation and cancer cytotoxicity of the T cells while still in culture ex vivo as well. The increased expression of IFN-γ may also be advantageous, as IFN-γ can induce apoptosis in cancer cells and drive increased activation of other immune cells including dendritic or natural killer cells (Jorgovanovic, D. et al. Biomark. Res. 2020 8:49). Additionally, IFN-γ has been shown to help T cells evade exhaustion induced by the PD-1 pathway (Dong, E. et al. Signal Transduct. Target. Ther. 2021 6:1-3). The role of TNF-α in cancer progression is complex, as it has been shown to induce cancer cell death, promote cancer cell survival, and increase T cell activation and proliferation (Mehta, A. K. et al. Cytokine. 2018 101:14-18; Jiang, Y. et al. J. Immunother. Cancer. 2019 7:28; Montfort, A. et al. Front. Immunol. 2019 10). However, in the ex vivo expansion of CAR T cells, these properties of TNF-α may be advantageous, as the TNF-α could promote the proliferation of T cells. TNF-α has also been shown to downregulate T-regulatory cell function in vivo, suggesting that the enhanced TNF-α expression could also help reduce T cell exhaustion (Valencia, X. et al. Blood. 2006 108:253).

Activating T cells through FSS could have other benefits for CAR T cells production. For example, the magnetic beads that are currently used for ex vivo T cell activation have been shown to less effectively activate CD8-positive T cells compared to CD4-positive T cells (Li, Y. et al. J. Transl. Med. 2010 8:104). The results of this study indicate that FSS is effective in activating both subpopulations of T cells (FIG. 4). Another limitation of CAR T cell therapy is the expensive cost of treatment, with a single treatment costing as much as $475,000 (Lin, J. K. et al. J Clin Oncol. 2019 37(24):2105-2119). A significant portion of these costs are due to the reagents needed for producing CAR T cells. FSS treatment of T cells via a cone-and-plate viscometer offers a potentially more scalable method for the ex vivo activation of T cells.

However, in the current work the T cells were only studied for a maximum of 24 h. If this method were to be used in T cell therapy production, it would be necessary to assay the T cells treated with FSS at later time points to measure how this affects their differentiation as TNF-α, IL-2 and IFN-γ are known to play significant roles in these pathways (Jiang, Y. et al. J. Immunother. Cancer. 2019 7:28; Zhu, J. et al. Annu. Rev. Immunol. 2010 28:445-489; Martinez-Sanchez, M. E. et al. Front. Physiol. 2018 9). Additionally, it would be of interest to measure how different FSS treatment regiments would affect T cell fate determination.

The mechanism of how FSS enhances T cell activation was also investigated (FIG. 5). It was found that the increase in ZAP70 phosphorylation was a calcium-mediated process, as removal of calcium either through chelation or a calcium-free buffer blocked the increased activation from FSS (FIG. 3). Additionally, events that are downstream of calcium signaling were inhibited to further establish the role of calcium influx in FSS activation of T cells. CCD was used to block actin polymerization, which caused a significant reduction in ZAP70 phosphorylation. Calcineurin was inhibited via CSA and this resulted in reduced NF-κB phosphorylation, suggesting that calcium also works downstream of ZAP70 phosphorylation (FIG. 3). GsMTx-4 and CRISPR/Cas9 knockout of Piezo1 were used to specifically determine if the mechanosensitive ion channel Piezo1 was involved in this process, as a recent study identified that the chemical agonist of Piezo1 could be used to boost T cell activation (Liu, C. S. C. et al. J. Immunol. 2018 200:1255-1260). Both methods of inhibition caused a decrease in ZAP70 phosphorylation in response to FSS treatment (FIG. 3). While this study identified Piezo1 as essential for FSS enhanced T cell activation, other mechanosensitive ion channels may also play roles in this process. TRPV4 activation has previously been shown to increase T cell activation through a calcium-mediated pathway, and TRPV4 is activated by FSS (Majhi, R. K. et al. FEBS J. 2015 282:2661-2681; Baratchi, S. et al. Sci. Rep. 2017 7:15942; Resnick, N. et al. Prog. Biophys. Mol. Biol. 2003 81:177-199). Additionally, Piezo1 has been implicated in having a pro-stimulatory effect on dendritic cells and macrophages, suggesting FSS could also have a pro-stimulatory effects in these cells as well (Solis, A. G. et al. Nature. 2019 573:69-74; Chakraborty, M. et al. Cell Rep. 2021 34:108609).

The present study focused on identifying if FSS could be used to enhance T cell activation for the production of T cell therapies. However, the results could also be applied to autoimmune diseases. Increasing the magnitude of FSS further increased T cell activation as measured by ZAP70 phosphorylation (FIG. 1). Hypertension is associated with increased blood flow velocity, and therefore increased FSS, in certain regions of the body, such as in the left and right common carotid artery, and coronary artery (Perret, R. S. et al. Ultrasound Med. Biol. 2000 26:1387-1391; Maria, L et al. J. Am. Heart Assoc. 6:e005710). Hypertension also presents increased hazard ratios with multiple autoimmune disorders, such as type I diabetes, multiple sclerosis, and rheumatoid arthritis (Han, C. et al. J. Rheumatol. 2006 33:2167-2172; Briggs, F. B. S. et al. Eur. J. Neurol. 2021 28:558-566; Cheung, B. M. Y. et al. Curr. Atheroscler. Rep. 2012 14:160-166). In each of these diseases T cells are known to play a prominent role (Raphael, I. et al. Cytokine. 2015 74:5-17). IFN-γ, which was upregulated by FSS in the present work, is known to further exacerbate autoimmune diseases, such as multiple sclerosis (Panitch, H. S. et al. Neurology. 1987 37:1097-1102; Lees, J. R. et al. J. Clin. Invest. 2007 117:297-299). Severe cytokine release syndrome by CAR T cell therapy and COVID-19 have also been shown to occur more frequently in hypertensive patients (Hong, R. et al. Bone Marrow Transplant. 2021 56:570-580). The results of this study and the correlation between hypertension and autoimmune diseases suggest that inhibition of Piezol and other mechanosensitive ion channels in T cells may hold therapeutic potential for autoimmune diseases and cytokine release syndromes.

### Materials & Methods

Reagents and Antibodies: RPMI 1640 cell culture media, fetal bovine serum (FBS), and penicillin streptomycin were obtained from Invitrogen. Bovine serum albumin (BSA), dimethyl sulfoxide (DMSO), Triton, and CSA were obtained from Sigma. HBSS with calcium and magnesium, and HBSS without calcium and magnesium were purchased from Gibco. GsMTx-4 was purchased from Abcam. CCD was obtained from Tocris. Ficoll-Paque was purchased from GE Healthcare. CRISPRMAX was purchased from ThermoFisher Scientific. Lamelli buffer was purchased from Bio-Rad. IRDye 8000 W and IRDye 680RD were obtained from LI-COR. 32% Paraformaldehyde was purchased from Electron Microscopy Sciences. Anti-human functional CD3 (OKT3), functional CD28 (CD28.2), FITC-CD69 (FN50), APC-CD25 (CD25-4E3), FITC-TNF-α (MA11), PE-TNF-α (MAb11), FITC-IL-2 (MQ1-17H12), PE-IL-2 (MQ1-17H12), FITC-IFN-γ (4S.B3), PE-IFN-γ (4S.B3), PE-phospho-ZAP70 (n3kobu5), PE-phospho-cFos (cFosS32-BA9), PE-phospho-NF-κB (B33B4WP), APC-CD4 (RPA-T4), FITC-CD8 (RPA-T8), APC-CD3 (OKT3), APC-CD3-zeta (6B10.2), and PE-ZAP70 (1E7.2) were purchased from eBioscience. Anti-human Piezol (15939-1-AP) was purchased from Proteintech. Anti-human GAPDH (MAB374) was purchased from EMD Millipore. Anti-human NFATc1 (7A6) was purchased from Biolegend. ActinRed 555 and DAPI were purchased from Molecular Probes. Annexin V and propidium iodide were obtained from BD Pharmingen.

Cell Culture: Jurkat cells were obtained from the ATCC. Jurkat cells were cultured in RPMI 1640 supplemented with 10% FBS and 1% penicillin streptomycin in an incubator at 37° C. and 5% CO₂.

Primary T cell samples: Primary T cells were isolated from healthy human volunteers after informed consent. On the day of activation, blood was collected in BD Vacutainer plastic blood collection tubes with sodium citrate. Peripheral blood mononucleocytes were purified from blood using ficoll gradient centrifugation. Following gradient centrifugation, T cells were isolated by using the Miltenyi Biotech human pan T cell isolation kit according to the manufacturer's instructions. After incubation with the antibodies targeting all mononucleocytes except for T cells and magnetic beads in the kit, the mononucleocytes were flowed through a magnetic column. The flow through was collected to isolate the purified T cell population. Purified T cells were resuspended in RPMI 1640 supplemented with 10% FBS and 1% penicillin streptomycin. All experiments with human primary samples were approved by Vanderbilt University's Institutional Review Board.

T cell activation by FSS: Jurkat or primary T cells were collected and resuspended at a concentration of 200,000 cells/mL in complete RPMI 1640 media. The cells were treated with or without functional grade antibodies targeting CD3 (OKT3) and CD28 (CD28.2) at a concentration of 2 μg/mL for both antibodies. The T cells were then loaded into a Brookfield cone-and-plate viscometer for FSS treatment as described previously in Mitchell & King (Mitchell, M. J. et al. New J. Phys. 2013 15:015008). In a cone-and-plate viscometer the FSS magnitude is equal in all radial locations of the viscometer. The shear rate (G) is defined by the equation:

$$G = \frac{ω}{tan ( θ )}$$

where ω is the angular velocity (rad/s) and θ is the angle of the cone (rad). The flow field was assumed to be laminar, and the fluid assumed to be Newtonian. Therefore, the FSS (τ) is governed by the equation:

τ=μG
where μ is the viscosity (cP) of the fluid. In these experiments, viscosity was approximately 2.5 cP. Before FSS treatment, the cone-and-plate viscometer was cleaned thoroughly with 70% ethanol. The stationary plate and the rotating cone were then incubated with 5% BSA for 1 h to block nonspecific adhesion. The cells were exposed to fluid shear stress ranging from 0.5-5.0 dyn/cm² for times ranging from 15 to 60 min. Primarily, the cells were treated with FSS of magnitude 5.0 dyn/cm² for 1 h.

Where indicated, Jurkat cells were pretreated with different compounds prior to antibody and FSS treatment. The Jurkat cells were incubated with either 5 μM Cyclosporin A (CSA), 10 μM Cytochalasin D (CCD), 10 μM GsMTx-4, or 2 mM EGTA 30 min prior to antibody and FSS treatment.

After FSS treatment, the cells were either stained immediately or cultured overnight in an incubator at 37° C. with 5% CO₂.

Flow cytometry antibody staining: When staining for intracellular proteins, the cells were immediately fixed with 4% paraformaldehyde for 10 min. The cells were washed thoroughly and permeabilized with 100% ice cold methanol for 10 min. After washing the cells, the cells were stained with the indicated fluorescent-tagged antibodies for 15 min in 1% BSA. The fluorescence of each sample was measured using a Gauva easyCyte 5HT flow cytometer. Gating was performed using FlowJo software. For Jurkat samples, debris was gated out and fluorescence was quantified using FlowJo software.

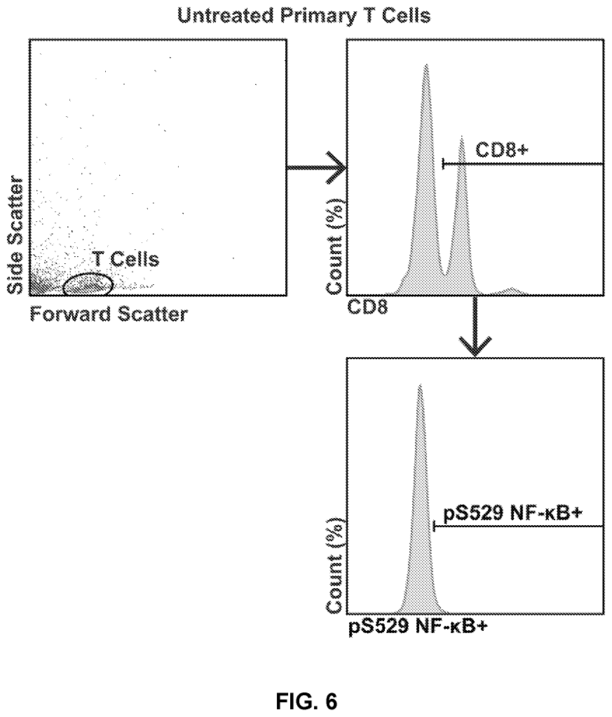

For primary T cell samples, after gating out cell debris, the CD4 and CD8 subpopulations were determined using fluorescence gating as shown in FIG. 6.

For intracellular cytokine staining, the cells were allowed incubate overnight following antibody and FSS treatment. 4 h prior to fixation, cells were treated with Golgiplug at 1:1000 ratio to block the secretion of cytokines. The cells were then stained using the same procedure as done for other intracellular proteins.

When staining for extracellular proteins such as CD69 and CD25, the cells were incubated with the indicated antibodies for 15 min in 1% BSA. The data collection and analysis were done following the same method as used for the intracellular proteins.

Confocal microscopy: Immediately after 1 h of FSS and antibody treatment, Jurkat cells were cytospun onto glass slides. The cells were fixed with 4% paraformaldehyde and permeabilized with 1% Triton. The cells were blocked with 5% BSA and stained overnight at 4° C. with antibodies against NFATc1. After staining for NFATc1 and washing, the cells were stained with an Alex Fluor 488 goat anti-rabbit secondary antibody, DAPI, and ActinRed 555 for 30 min. The cells were then imaged using a Zeiss LSM800 confocal microscope with a 40×/1.1NA water immersion objective. Image analysis was performed with ImageJ software. NFAT activation was measured using the fluorescent colocalization of the NFATc1 antibody and DAPI. The colocalization was quantified by calculating the area per cell where a cell was positive for both NFATc1 and DAPI fluorescence.

FRET: After T cells were treated with FSS and antibodies for 1 h, the cells were stained with PE-ZAP70 and APC-CD3-zeta for FRET analysis by flow cytometry using the protocol described in Ujlaky-Nagy et al (Ujlaky-Nagy, L. et al, in Flow Cytometry Protocols, T. S. Hawley, R. G. Hawley, Eds. (Springer, New York, NY, 2018, Methods in Molecular Biology, pp. 393-419). FRET efficiency (E) was calculated using the equation:

$$E = 1 - \frac{{I}_{D o n o r + A c ceptor} - {I}_{Unstained}}{{I}_{Donor only} - {I}_{Unstained}}$$

where I is fluorescence intensity of the donor fluorophore (RFU), in this case, the PE-ZAP70. I_{Donor+Acceptor} is the fluorescence intensity of a treated sample, such as the FSS-antibody treated group or the antibody-only treated group, stained with both the donor and acceptor antibodies. I_{Unstained} is the fluorescence intensity of an unstained control. I_{Donor} is the fluorescence intensity of a control group stained with only the donor antibody, PE-ZAP70.

Piezo1 knockout: CRISPR/Cas9 knockout was carried out using the Synthego Gene Knockout Kit V2 with sgRNAs targeting human Piezol. The Jurkat cells were transfected for 3 days following the Synthego kit's instructions for CRISPRMax-based knockout.

Western Blot: Jurkat cells were lysed in Laemelli buffer. The lysates were resolved by SDS-PAGE electrophoresis, and then transferred to PVDF membranes. The membranes were blocked with 5% BSA in TBS-Tween. The membranes incubated overnight at 4° C. with primary antibodies against human Piezol and GAPDH. The membranes were then washed and stained with IRDye LI-COR fluorescent secondary antibodies. The membranes were imaged using the Odyssey CLx. The membrane fluorescence intensities were quantified using the Image Studio Lite software.

Annexin V: After Jurkat cells were treated with FSS for 1 h, the cells were washed with HBSS. The cells were then stained for 15 min with Annexin V and propidium iodide according to the manufacturer's directions in HBSS with calcium and magnesium buffer. After staining, the cells were collected using flow cytometry. Cells negative for both Annexin V and propidium iodide were identified as being viable. Analysis was done in FlowJo.

Statistics: Results are presented as means and standard deviation (SD). Statistical comparisons for Jurkat cells were done using unpaired Student's t tests. Statistical comparisons for primary T cells were done using paired t tests. Linear regression was used to determine if the slopes significantly deviated from zero. Each experiment included at least 3 independent trials. P<0.05 was used as the threshold for determining statistical significance. GraphPad Prism 8 software was used to prepare figures and perform statistical comparisons.

### Example 2: Fluid Shear Stress Enhances Dendritic Cell Activation

### Introduction

Therapeutics have been developed in which a patient's dendritic cells (DCs) are removed, activated ex vivo, and reintroduced into the body to enhance the efficacy of treatments (Y Gu, APS, 2020, 41, 959-969; A Huber, Front Immunol, 2018, 9, 2804; Crews, Front Immunol, 2021, 11, 626463). The first FDA-approved therapeutic cancer vaccine, Sipuleucel-T (Provenge), was approved in 2010 for the treatment of metastatic castration-resistant prostate cancer (mCRPC) (MA Cheever, Clin Cancer Res, 2011, 17; E Anassi, P T, 2011, 36, 197-202). For this therapy, DCs are activated with granulocyte macrophage-colony stimulating factor (GM-CSF) or prostatic acid phosphatase (PAP), facilitating T cell priming and allowing for targeting of prostate cancer cells (MA Cheever, Clin Cancer Res, 2011, 17; E Anassi, P T, 2011, 36, 197-202). While Sipuleucel-T is associated with low treatment periods as well as low hospitalization rate due to adverse events, the cost of the therapeutic is expensive to patients (E Anassi, P T, 2011, 36, 197-202).

Internal and external forces play a role in activating a variety of cell types, ranging from endothelial cells and immune cells to cancer cells (C R White, Phil Trans R Soc B, 2007, 362; R M Narem, ASGSB Bull, 1991, 4, 87-94; K Yamamoto, Circ Res, 2000, 87). Shear stress is a force which activates cells by initiating membrane deformation, resulting in the opening of mechanosensitive ion channels (MSCs) such as Piezo1, which affects signal transduction through an influx of calcium ions (J M Hope, Cell Death & Dis, 2019, 10, 1-15; D A Chistiakov, Acta Physiologica, 2016, 219, 382-408; J Rossy, Front Immunol, 2018, 9, 2638; E S Haswell, Structure, 2011, 19; B Martinac, 2004, 117; D de Felice, Cancers, 2020, 12). Calcium is a ubiquitous secondary messenger responsible for a host of intracellular responses (K Yamamoto, Circ Res, 2000, 87; K A Gerhold, Physiology, 2016, 31; J Ando, C V R, 2013, 99; S M Swain, bioTxiv, 2020, 1-32; L Sang, Mol Cell, 2018, 72). Fluid shear stress (FSS) is a force which many immune cells experience due to blood flow in circulation (H J Lee, Nat Com, 2017, 8; F Moazzam, PNAS, 1997, 94; D L Harrison, Front Phys, 2019, 7). Several studies have demonstrated that immune cells such as T cells and platelets are sensitive to shear stress, with increased proliferation and cytokine production, respectively (CSC Liu, J I, 2018, 200; J Rossy, Front Immunol, 2018, 9; M Hagihara, J I, 2004, 172). Specifically, shear stress promotes maturation, growth, and progression of the cell cycle in DCs (E Shumilina, Am J Physiol Cell Physiol, 2011, 300, C1205-1214). One study applied cyclic strain to DCs and observed a resulting increase in expression of MHC II and co-stimulatory molecules, although there has not been significant research into D C activation via shear stress (J S Lewis, Biomaterials, 2013, 34).

FSS can be recreated in vitro via cone-and-plate or other flow devices (MJ Mitchell, N J P, 2013, 15; MJ Mitchell, PNAS, 2014, 111, 930-935; N Jyotsana, Science Advances, 2019, 5, eaaw4197). Cone-and-plate flow devices are advantageous in that, due to the shape, they apply the same shear rate to cells regardless of position within the device (MJ Mitchell, N J P, 2013, 15). In this study, DCs were stimulated with FSS applied via cone-and-plate flow device, analyzing various markers of activation.

### Results

Shear Stress Enhances NF-κB Phosphorylation in DCs after 1 h

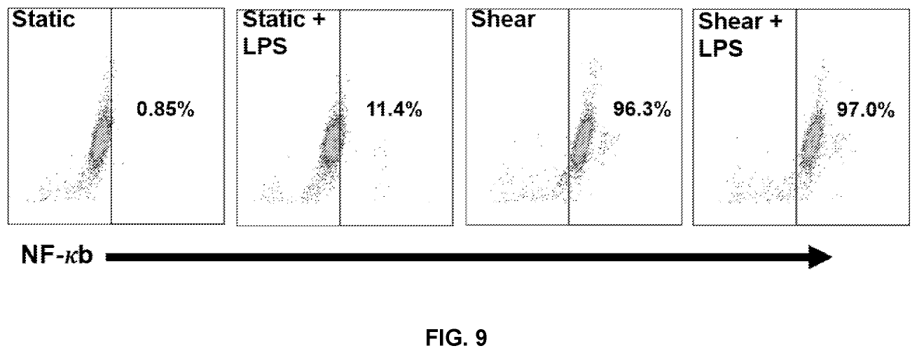

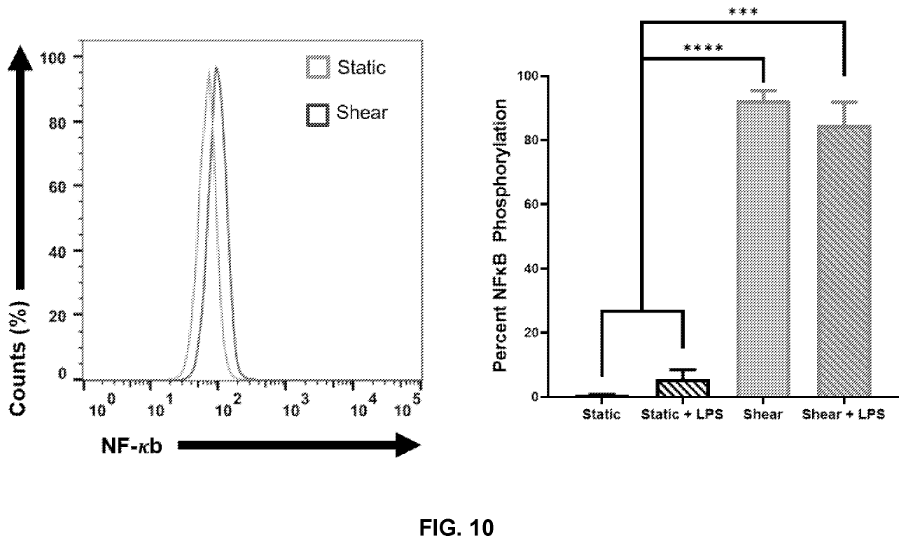

Nuclear factor-kappa B (NF-κB) phosphorylation was measured in DCs to analyze activation. NF-κB transcription factor is regulated by calcium influx, and leads to increased cytokine expression, maturation, survival, and cell cycle progression in a variety of cell types including DCs (T Liu, Sig Transduct Target Ther, 2017, 2; B Vuong, J Neuroinflammation, 2015, 12; E C Dresselhaus, K I, 1998, 54; F Ouaaz, Immunity, 2002, 16; M Rescigno, J Exp Med, 1998, 188; N Ade, Tox Studies, 2007, 99). DC2.4 cells exposed to FSS via cone-and-plate viscometer for 1 h at 5.0 dyn/cm² FSS were observed to have a significant shift in NF-κB phosphorylation compared to static controls when expression was analyzed via flow cytometry (FIGS. 9-10). There was no significant difference in phosphorylation observed between FSS with lipopolysaccharide (LPS) as a potent stimulator and FSS without LPS.

AP-1 Expression in DCs is Regulated by FSS after 1 h

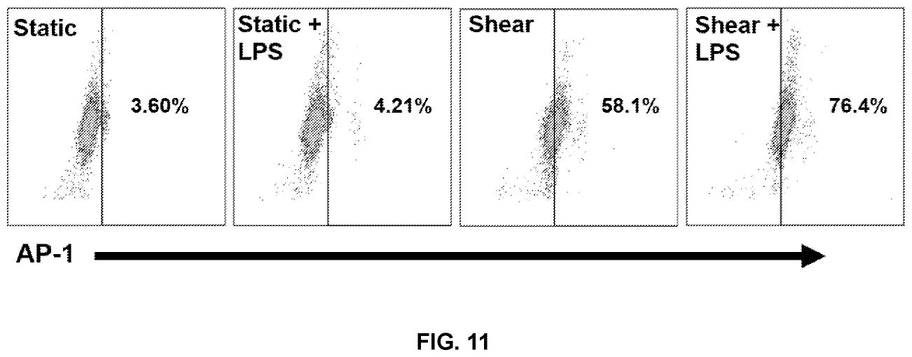

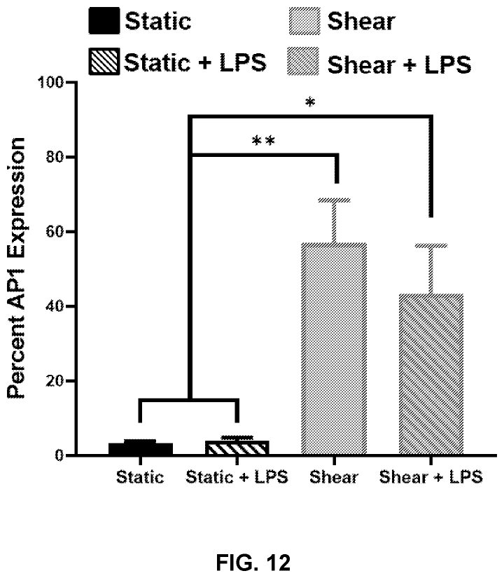

Activator protein 1 (AP-1) expression was analyzed for this study. AP-1 is another transcription factor that is regulated by calcium ions and responsible for controlling cellular processes such as proliferation and regulation of cytokine release (E Shaulian, Oncogene, 2001, 20, 2390-2400; V Atsaves, Cancers, 2019, 11, 1037). DC2.4 cells were stimulated with 5.0 dyn/cm² FSS via cone-and-plate flow device for 1 h. A shift in AP-1 expression was observed for the shear conditions, although less significant than for NF-κB phosphorylation (FIGS. 11-12). As with data observed for NF-κB phosphorylation, there was not a significant difference in AP-1 expression between shear conditions.

### Potent Stimulator LPS Plays a Significant Role in MHC and Costimulatory Molecule Expression

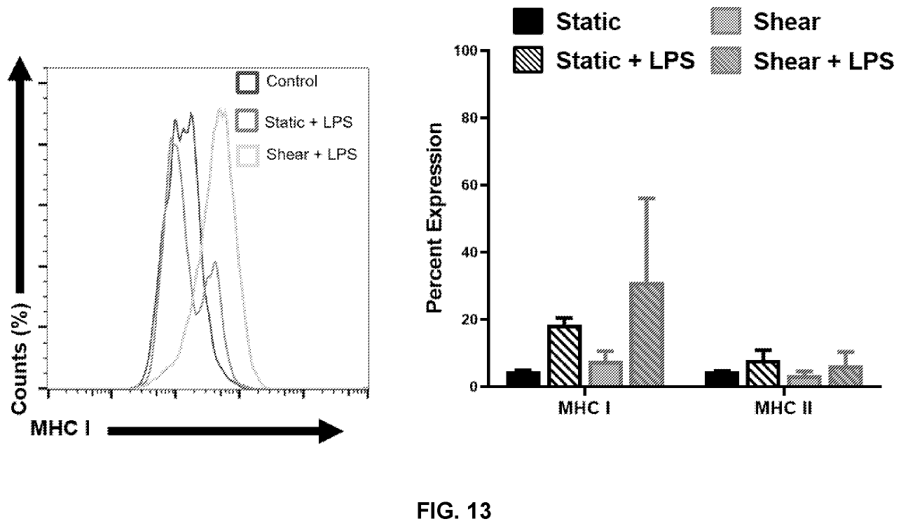

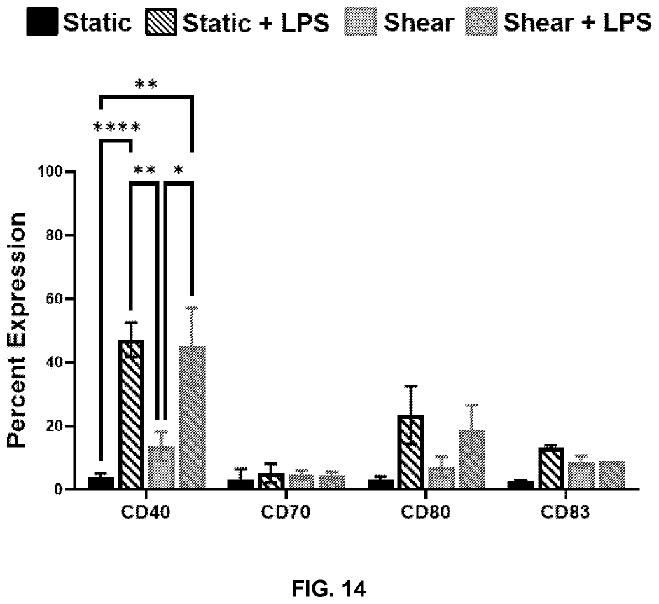

Following 1 h stimulation via cone-and-plate flow device at 5.0 dyn/cm² FSS, DC2.4 cells were plated and incubated at 37° C. for 24 h to look at later markers of activation. Major histocompatibility complexes (MHCs) and costimulatory molecules such as CD40, CD70, CD80, and CD83 signify DCs acting as antigen-presenting cells (APCs) to facilitate T cell priming (S Fujii, J Exp Med, 2004, 199, 1607-1618; S Dilioglou, Exp Mol Pathol, 75, 217-227; J Sprent, Current Biology, 1995, 5, P1095-1097). After 24 h, cells were analyzed for the presence of MHCs and costimulatory molecules. There was not a significant increase in MHC expression with DC stimulation via FSS (FIG. 13). Most interestingly, costimulatory molecule expression was enhanced in the presence of LPS, regardless of whether shear stress was present (FIG. 14). This trend is observed for CD80, and significant for CD40. The results indicate the importance of an activating factor if a specific T cell response is desired.

DC Cytokine Production was Enhanced with 1 h FSS

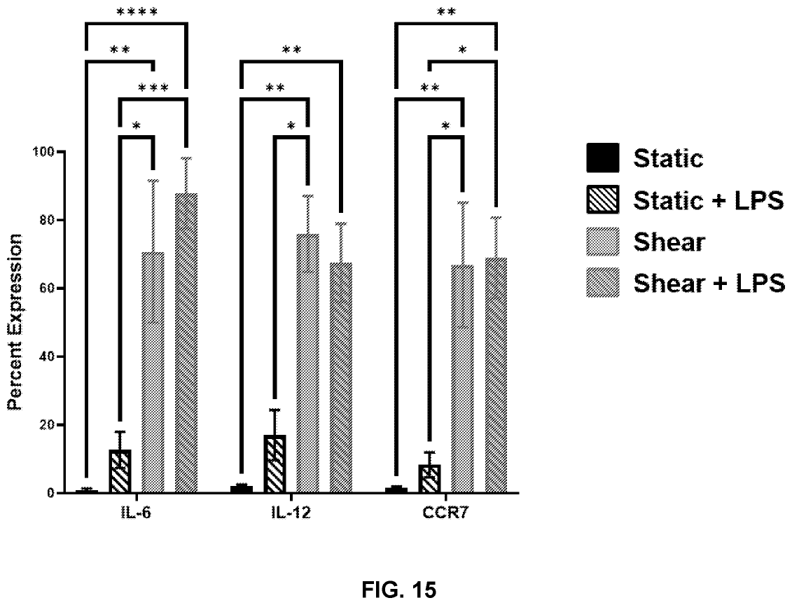

DC2.4 cells were plated for 24 h following stimulation via 5.0 dyn/cm² FSS for 1 h. Cytokines and chemokines are a direct measure of immune cell activation, especially for DCs, where they aid in trafficking and driving specific T cell activation (P Blanco, Cytokine Growth Factor Rev, 2008, 19, 41-52). There was a significant increase in release of IL-6, IL-12 and CCR7 for FSS-activated DCs compared to static controls (FIG. 15). There was not a significant difference between FSS and FSS with LPS.

### Transcription Factor NF-κB has a Major Role in Cytokine Release for FSS-Activated DCs

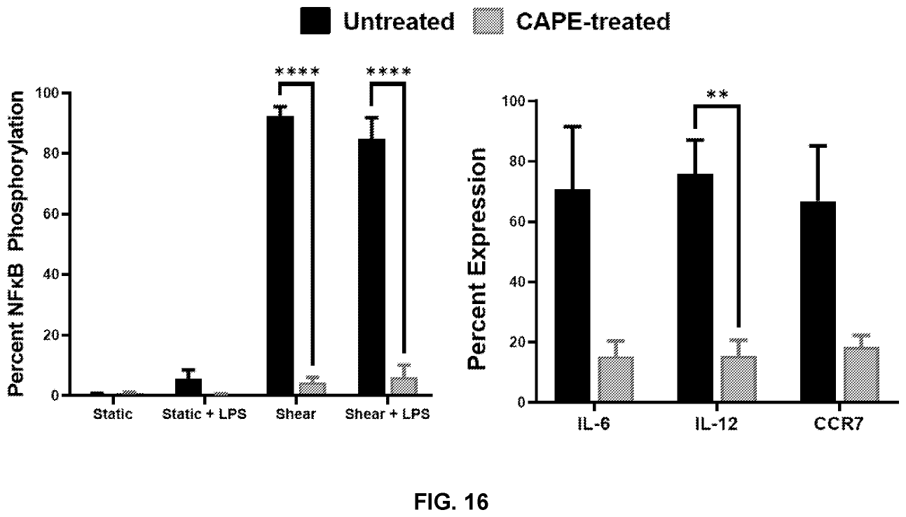

The effect of NF-κB phosphorylation on cytokine release was investigated using caffeic acid phenethyl ester (CAPE), which inhibits NF-κB phosphorylation by preventing the translocation of the p65 subunit of NF-κB to the nucleus (K Natarajan, PNAS, 1996, 93, 9090-9095). The effect of CAPE on NF-κB phosphorylation was first analyzed by comparing NF-κB phosphorylation with CAPE treatment to untreated DCs. CAPE-treated cells were observed to have a significant reduction in NF-κB phosphorylation compared to untreated cells (FIG. 16). When cytokine release after CAPE treatment was investigated after 24 h, a significant reduction was observed in IL-12 release, as well as a strong trend in the reduction of IL-6 and CCR7 production (FIG. 16).

### Discussion

This study demonstrated that FSS activates DCs in vitro, while also beginning to explore the mechanism behind this activation. With just 1 h of applied FSS, major cellular changes were seen to signify DC activation. NF-κB phosphorylation and AP-1 expression were enhanced with stimulation via FSS. Increased expression of these transcription factors gives insight into the pathways involved in DC activation via FSS. These transcription factors play a major role in DC function and maturation, particularly through the release of cytokines such as IL-6, IL-12 and CCR7. DCs stimulated with FSS were observed to have enhanced release of all measured cytokines and chemokines compared to static controls. The release of these factors is significant to the success of DCs as APCs. To determine the extent to which NF-κB plays a role in DC activation via cytokine release, DCs were treated with the inhibitor CAPE. After having observed a sharp and significant reduction in NF-κB phosphorylation, it was concluded that the inhibitor was effectively preventing this activation. Cytokine release studies found that all analyzed cytokines and chemokines had significantly reduced levels of production after DCs were treated with CAPE. This reduction indicates that NF-κB is likely the major pathway by which DCs are being activated by FSS.

While there was not a significant difference in MHC and costimulatory molecule expression in DCs with FSS stimulation, there was a significant enhancement in the presence of the potent stimulator LPS, particularly for CD40. The results signify that, if enhanced costimulatory molecule expression is desired, a stimulator is necessary in addition to the fluid shear stress treatments. Perhaps incubation with a tumor-specific antigen (TSA) or tumor-associated antigen (TAA) could aid in achieving this specific response; for instance, PAP could be used in mCRP therapies (H Westdorp, Front Immunol, 2014, 5, 191).

Despite the major advances in ex vivo therapeutics, the cost of these treatments remains high for patients. Finding a way to reduce these costs while further enhancing activation can be advantageous to developing successful therapies. The results from this study suggest that FSS could be used in the production of DC-based immunotherapies to drive down costs of these treatments. Through the ex vivo stimulation of DCs via FSS in combination with an additional activating factor, the necessary transcription factors can be activated, cytokines released, and costimulatory molecules enhanced, in order to produce successful therapeutics.

### Materials and Methods

Cell culture: DC2.4 murine DCs were purchased from Sigma-Aldrich (Catalog No. SCC142). DCs were grown in culture in media consisting of RPMI-1640 supplemented with 10% fetal bovine serum (FBS), 1×L-Glutamine, 1× non-essential amino acids, 1×HEPES buffer solution and 0.0054× β-Mercaptoethanol, as recommended by the supplier's Product Data Sheet. Cells were maintained in an incubator at 37° C. with 5% CO₂, with experiments performed at ˜80% confluency.

Reagents: RPMI-1640 and FBS (Invitrogen), non-essential amino acids and HEPES buffer (Gibco), and β-Mercaptoethanol (Sigma-Aldrich) were obtained for preparation of the DC2.4 cell culture media. Hank's balanced salt solution (HBSS) with and without calcium and magnesium was purchased from Gibco. Lipopolysaccharide (LPS) solution (500×) was purchased from Thermo Scientific. 32% paraformaldehyde aqueous solution, electron microscopy grade was obtained from Electron Microscopy Sciences. Caffeic acid phenethyl ester (CAPE) was obtained from Tocris. Anti-mouse PE-MHC (Class I H-2Kk) was purchased from Antibodies-Online.com. Anti-mouse PE-CD40 (5C3) was purchased from BioLegend. Anti-mouse PE-CD70 (FR70), PE-CD80 (16-10A1), PE-1 A/I E (M5/114.15.2) (MHC II), PE-IL-6 (MP5 20F3), and PE-IL-12 (p40/p70) (C15.6) were obtained from BD. Anti-mouse PE-CD83 (Michel 17), PE-CD197 (CCR7) (4B12), PE-Phospho-NFkB p65 (Ser529) (NFkBp65S529-H3), and PE-Phospho-c-Fos (Ser32) (cFosS32-BA9) were obtained from Thermo Fisher Scientific.

Fluid shear stress application: A Brookfield cone-and-plate viscometer, which consists of a stationary plate and rotating cone, was used to apply FSS to DCs throughout this study. The protocol used for these experiments was described previously (M J Mitchell, N J P, 2013, 15). The advantage of using a cone-and-plate flow device for applying FSS is that the cells experience the same shear rate in all locations in the device. Flow is assumed to be laminar, and the fluid is assumed to be Newtonian. Therefore, a series of equations could be used to determine important physical properties. The equations are as follows:

$$G = \frac{ω}{tan ( θ )} ,$$

where G is a shear rate, ω is angular velocity (rad/s), and θ is angle of the cone (rad); and
τ=μG, where τ is the FSS and μ is the viscosity (cP). μ is approximately 2.5 cP during experiments.

Prior to prepping cells, the cone-and-plate was incubated with 5% bovine serum albumin (BSA) for 1 h in order to block non-specific binding. DC2.4 cells were lifted with trypsin, washed, and resuspended in complete RMPI media at 200,000 cells/mL. Cells were placed on a rotator for static conditions. For shear conditions, an FSS of 5.0 dyn/cm² was applied for 1 h using cone-and-plate flow device. Static and shear conditions were either left untreated or treated with lipopolysaccharide (LPS) potent stimulator at 10 μg/mL.

Flow cytometry analysis and antibody staining: Following FSS stimulation, cells were plated overnight or immediately prepped for flow cytometry analysis. For intracellular proteins such as NF-κB and AP-1, cells were fixed with 4% paraformaldehyde for 10 min. Cells were washed with HBSS and permeabilized with 100% ice cold methanol. Cells were washed again and stained for 15 min in the dark with antibodies fluorescently-labeled with PE fluorophore while suspended in 1% BSA. Guava easyCyte 5HT flow cytometer was used to measure fluorescent intensity, with FloJo software used for gating and analysis. For extracellular proteins analyzed after 24 h, cells were lifted with trypsin, washed, and incubated with fluorescently-labeled antibodies in 1% BSA and then washed a final time.

Cytokine staining: For intracellular cytokine staining (ICS), cells were plated overnight following FSS stimulation. After 24 h, cells were treated with 1:1000 GolgiPlug Transport Inhibitor to block cytokine secretion. After 4 h, cells were lifted and stained following the previously described protocol for staining intracellular proteins. IL-6, IL-12 and CCR7 were investigated in this study.

Inhibition of NF-κB: NF-κB was inhibited using caffeic acid phenethyl ester (CAPE). DC2.4 cells were lifted, washed and suspended in 200,000 cells/mL complete media. 1 μL of CAPE was added to this suspension. Cells were placed on a rotator for 30 min to allow for effective inhibition, and then added to the cone-and-plate viscometer for FSS stimulation. Following FSS, NF-κB phosphorylation analysis was performed after 1 h FSS. Following stimulation, 100,000 cells were plated overnight for cytokine analysis and 100,000 cells were immediately analyzed for NF-κB phosphorylation. NF-κB and cytokine assays were performed as described previously.

Statistics: All data are reported as mean and standard error of measurement. Statistics were determined using a student's T test unless otherwise indicated. Each experiment included a minimum of three independent replicates. Significance is indicated by *p<0.05, **p<0.01, ***p<0.005, and ****p<0.001. GraphPad Prism software was used to perform statistical comparisons and generate figures for this manuscript.

### Example 3. T Cell Activation is Enhanced with Fluid Shear Stress Stimulation

### Results

FSS Enhances and Retains T Cell Activation Signatures in Jurkat Cells Treated with and without CD3/CD28 Antibody Coated Beads.

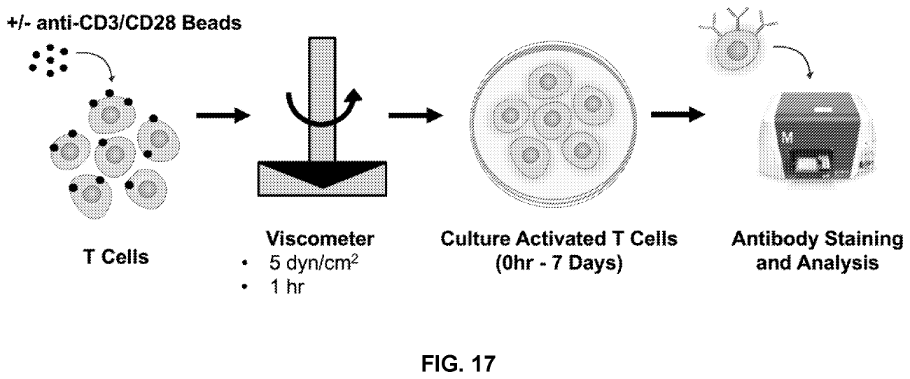

The immortalized Jurkat cell line was used to investigate the effects of FSS on T cell activation in the presence and absence of anti-CD3/CD28 antibody beads overtime in vitro. Jurkat cells were subject to combination treatments that included incubation with or without magnetic beads coated with antibodies against CD3/CD28 and with or without FSS exposure for 1 h. These combination treatments will be referred to as static (St1, no treatment), CD3/CD28 bead only treatment (St2), FSS only treatment (SS1), and FSS and CD3/CD28 bead combination treatment (SS2). Cone-and-plate viscometers were used to expose the cells to 5.0 dyn/cm² FSS. Following the activation treatments, T cells were cultured and analyzed at 0 hr, 48 hr, 5 days, and 7 days for indicators of T cell activation in flow cytometry (FIG. 17).

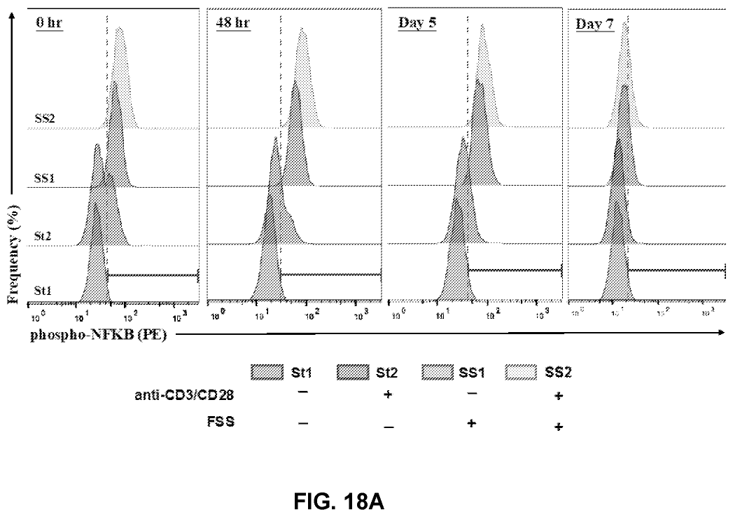

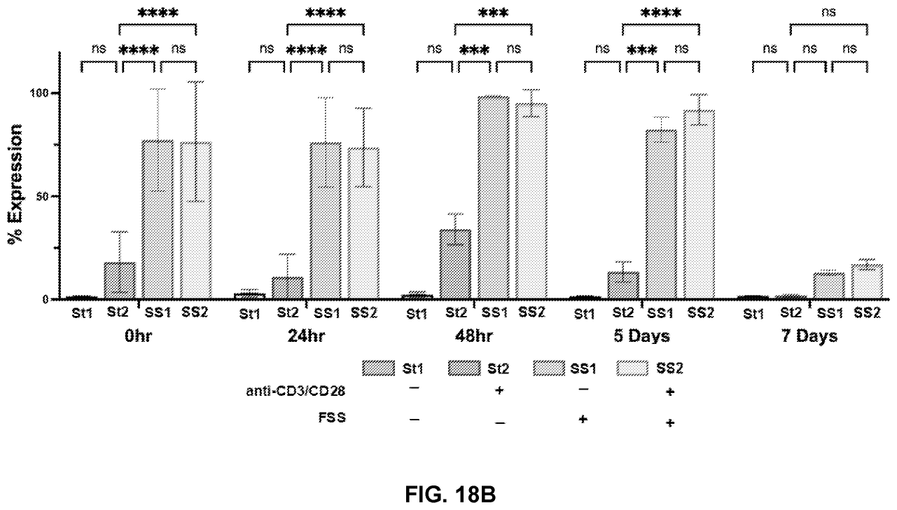

Both FSS treatment conditions significantly enhanced the phosphorylation of NF-kB compared to the untreated static condition and anti-CD3/CD38 bead only treatment (FIG. 18A, B). NF-kB activation is an important step in the T cell activation signaling pathway and is indicative of activation (Christian, F. et al. Cells. 2016 5). A rightward shift of the histograms obtained from flow cytometry show enhanced and sustained activation of NF-kB in the FSS treatment conditions compared to the static and CD3/CD28 treatment. There was a remarkable increase in NF-kB phosphorylation under the FSS conditions, where NF-kB activation peaked at 48 hours and subsided at day 7. No significant difference was observed between the static condition and the anti-CD3/CD28 bead treatment for any timepoint.

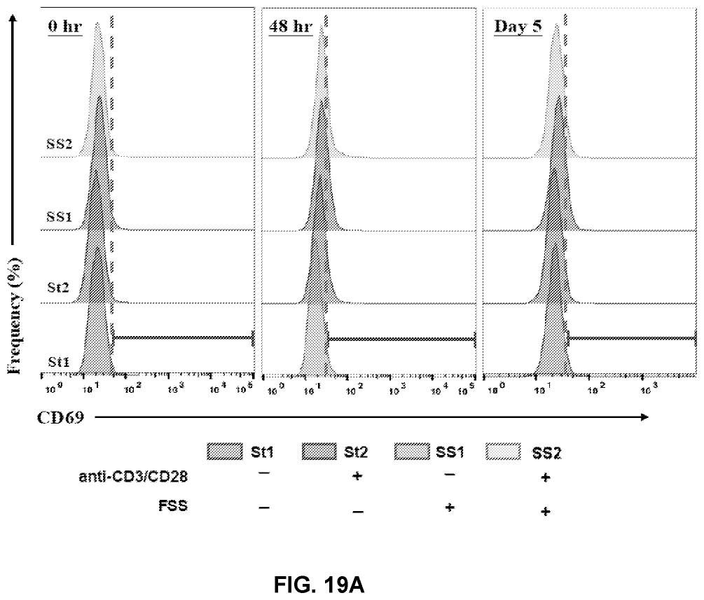

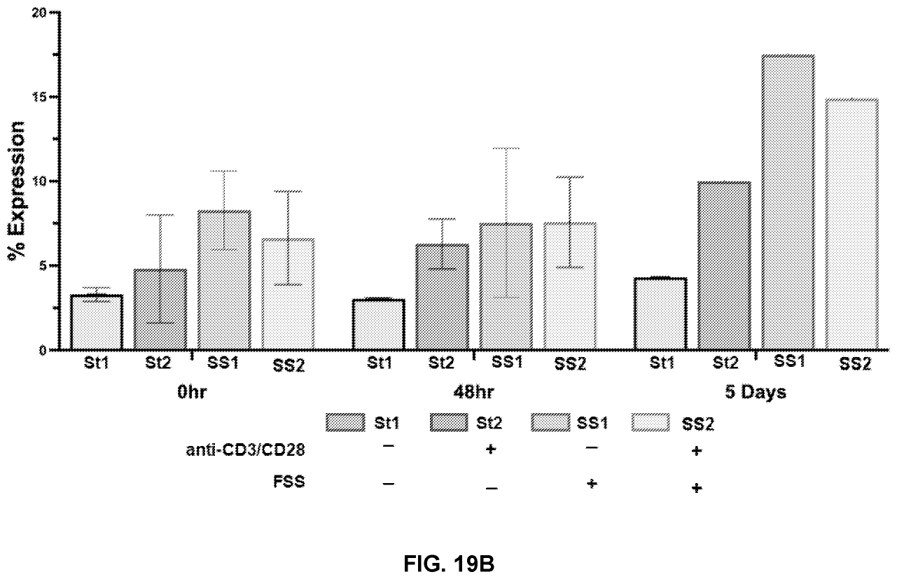

Expression of a later-stage activation marker, CD69, was enhanced at 5 days following FSS in the presence and absence of CD3/CD28 beads (FIG. 19A, B). As an activation marker on the surface of T cells, longer incubation times may be required to observe a significant upregulation of CD69 expression. Therefore, additional timepoint replicates are needed to quantify significance in CD69 expression over time. However, an increasing trend of CD69 expression was observed in both FSS treatment groups compared to the static and CD3/CD28 bead treatment groups, with the greatest increase in expression observed on day 5.

### FSS Enhances CAR T Cell Transduction in Jurkat and Primary Prostate Cancer Patient T Cells.

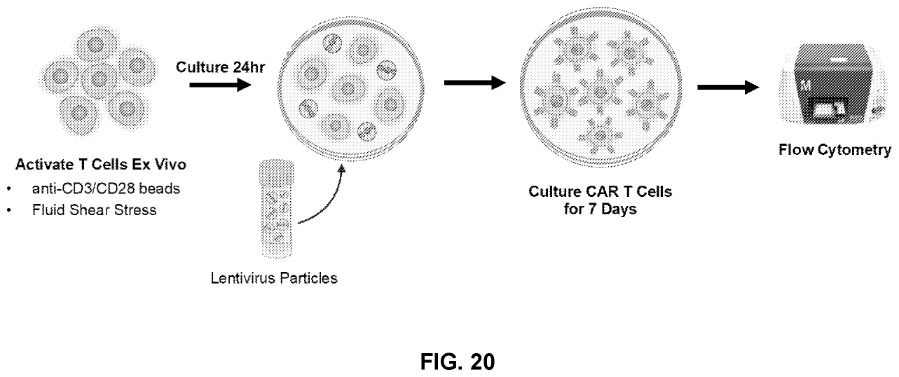

CAR T cell immunotherapy requires sufficient ex vivo activation of T cells prior to CAR transduction (Zhang, H. et al. Exp Hematol Oncol. 2020 9:34). Jurkat T cells and primary T cells isolated from a whole blood prostate cancer patient sample were activated ex vivo via combination treatments of FSS and anti-CD3/CD28 beads. After activation treatments, T cells were incubated for 24 hours, then cultured with lentiviral particles that encode for a prostate-specific membrane antigen (PSMA) targeting CAR construct (FIG. 20). T cells were cultured for 7 days following the addition of lentiviral particles and transduction efficiency was quantified with flow cytometry.

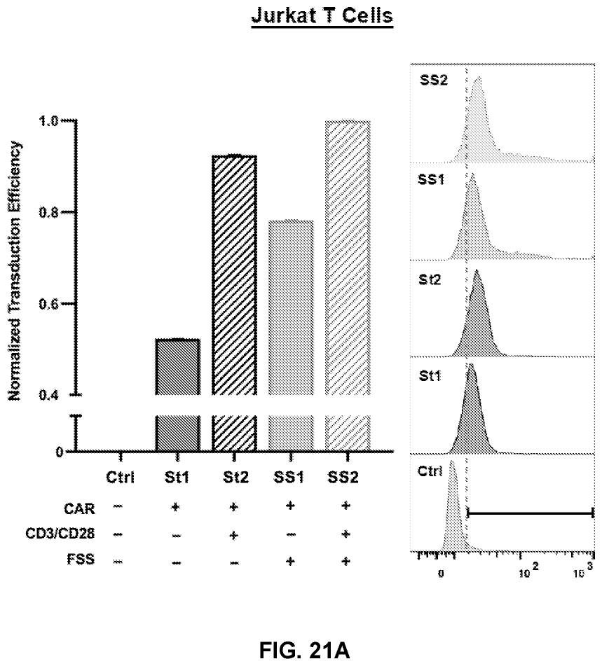

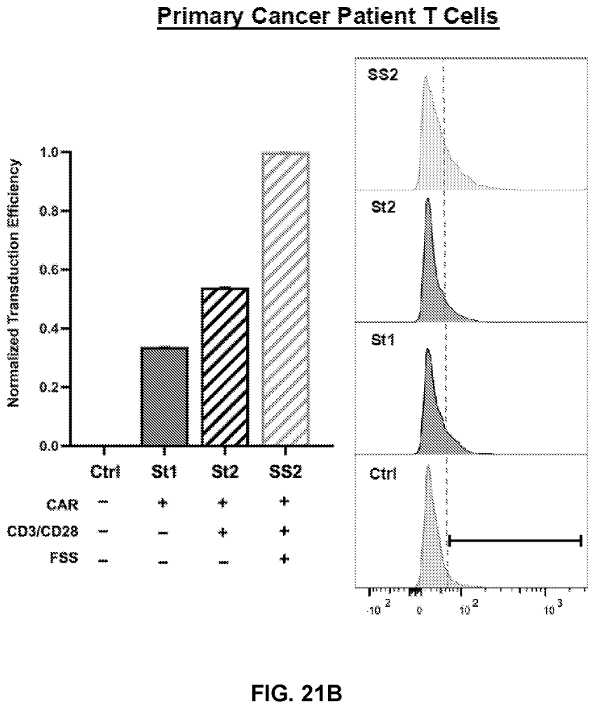

Both immortalized Jurkat CAR T cells and primary prostate cancer patient CAR T cells showed increased transduction efficiency following FSS and anti-CD3/CD28 bead treatment (FIG. 21A, B). Transduction efficiency was quantified by obtaining the Median Fluorescent Intensity (MFI) of each treatment group and normalizing it to the MFI of the control condition of T cells that was not transduced with the anti-PSMA CAR construct.

### FSS Enhances Primary Prostate Cancer Patient CAR T Cell Cytotoxicity.

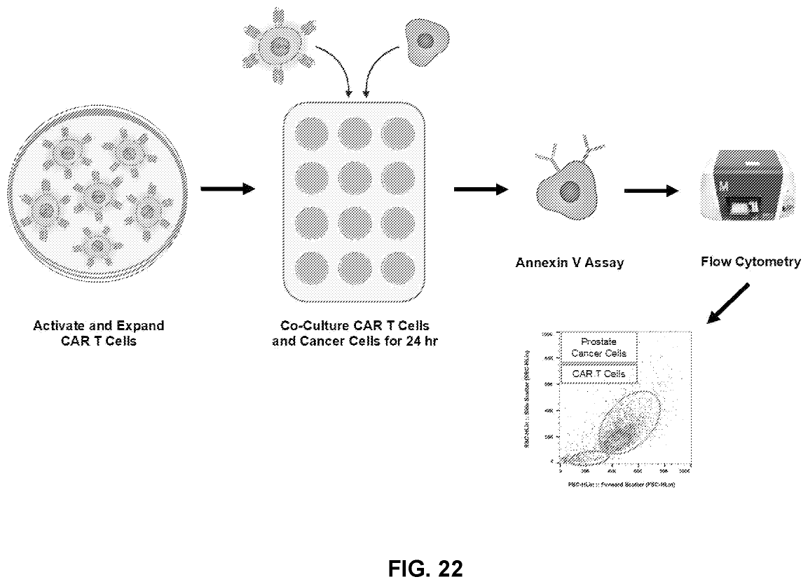

To determine the effects of FSS activation on T cell cytotoxicity, primary prostate cancer patient T cells were transduced with or without a PSMA-targeting CAR construct and activated with combination FSS and anti-CD3/CD28 bead treatment. Primary T cells and CAR T cells were incubated with prostate cancer cells (PC3) and cancer cell viability was evaluated after 24 hours using the Annexin V assay in flow cytometry (FIG. 22). The PC3 cell population was identified for analysis based on an increase in forward scatter and side scatter in flow cytometry plots, representing an increase in cell size and granularity, respectively. Furthermore, PC3 cells were incubated with a cell tracking dye 24 hours prior to the addition of CAR T cells to ensure that only PC3 cancer cells were analyzed.

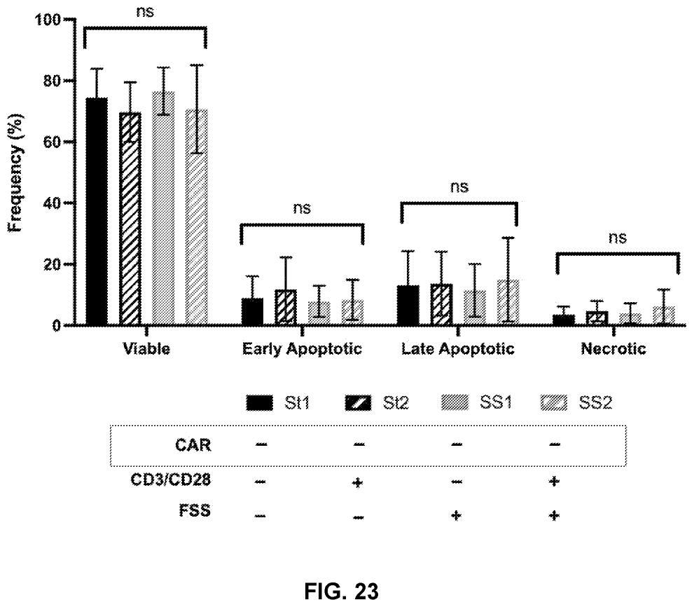

Primary T cells that were not transduced with a CAR construct exhibited no significant different in cytotoxicity against PC3 cells in any of the activation treatment groups (FIG. 23). PC3 cell viability following 24 hour co-culture with T cells was approximately 75% for the static and FSS-treated T cells, and approximately 70% for the CD3/CD28 bead treatment with or without FSS conditions. Therefore, the cytotoxicity of primary T cells is not affected by activation treatments in vitro.

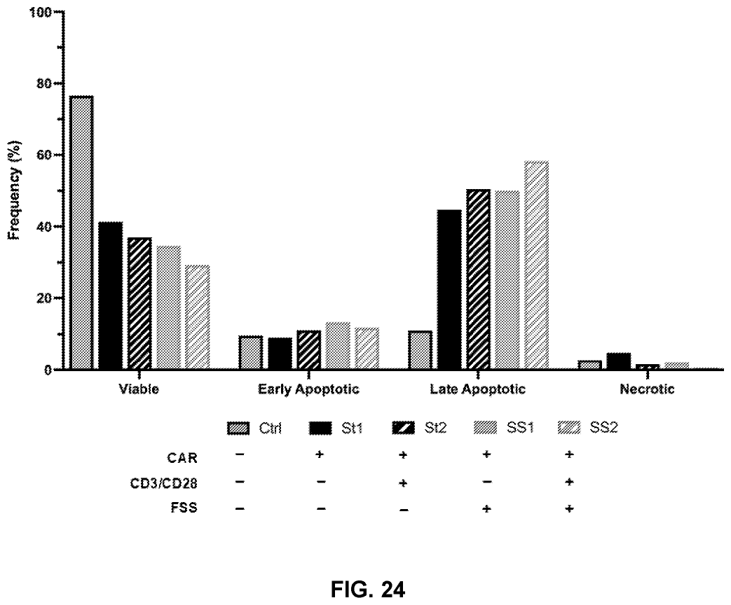

Primary prostate cancer patient T cells transduced with a PSMA-targeting CAR construct exhibited a trend of decreased PC3 cell viability in CAR T cells activated with FSS (FIG. 24). The addition of the anti-PSMA CAR construct on primary T cells resulted in a 35%-45% decrease in PC3 cell viability. Both of the FSS activated CAR T cell treatment groups showed increased cytotoxic effects against PC3 cells when compared to the static and anti-CD3/CD28 bead-treated CAR T cells.

### Discussion

T cell exposure to FSS in the presence and absence of anti-CD3/CD28 beads enhance T cell activation signatures in Jurkat T cells and lengthens the longevity of activation. FSS conditions increased phosphorylation of NF-kB (early indicator of activation) and expression of CD69 (later-stage surface marker of activation), where these increases were sustained for days following activation, compared to treatment groups that were not exposed to FSS. Calcium signaling and T cell activation levels dictate the downstream behavior of T cells, including proliferation, differentiation, and cytokine production (Joseph, N. et al. Biochimica et Biophysica Acta (BBA)-Biomembranes. 2014 1838:557-568). With the mechanisms identified here, linking FSS exposure to increased Piezo1 activation, subsequent calcium influx, and enhanced T cell activation over time, T cell exposure to FSS can be utilized and modified for the desired application. Furthermore, understanding the kinetics of increased T cell activation via FSS enables the optimization of experimental planning for any application involving ex vivo T cell activation, possessing strong clinical relevance, e.g. adoptive T cell therapies (CAR T cell therapy), and cellular engineering applications (T cell transfection/transduction).

T cell exposure to FSS in the presence of anti-CD3/CD28 antibody beads shows a promising trend for enhancing immortalized and primary PSMA targeting CAR T cell transduction. Additionally, primary prostate cancer patient anti-PSMA CAR T cells activated ex vivo via FSS and anti-CD3/CD28 beads exhibit a promising trend in increased cytotoxicity against PC3 prostate cancer cells. Although only one experimental replicate was obtained, the trend of enhanced transduction and cytotoxicity of FSS activated CAR T cells is extremely encouraging, as cancer patient immune cells are often impaired from cancer treatments and primary samples are often highly variable.

In the context of immuno-oncology, insufficient T cell activation is heavily responsible for the lack of clinical success of T cell immunotherapies (Saibil, S. D. & Ohashi, P. S., Curr Oncol. 2020 27:98-105). Therefore, the inexpensive and high-throughput approach to enhancing T cell activation via FSS holds strong clinical relevance, as this can be applied to any application requiring ex vivo T cell activation.

### Materials and Methods

Reagents and Antibodies: RPMI 1640 cell culture media, fetal bovine serum (FBS), and penicillin streptomycin were obtained from Invitrogen. HEPES Buffer Solution (1M) was purchased from R&D Systems and bovine serum albumin (BSA) was purchased from Sigma. HBSS with calcium and magnesium, and HBSS without calcium and magnesium were purchased from Gibco. Ficoll-Paque was purchased from GE Healthcare. Pan T cell isolation kit was purchased from Miltenyi Biotec. Human T-Activator CD3/CD28 Dynabeads™ and CellTracker™ Deep Red Dye were purchased from ThermoFisher Scientific. PE-phospho-NF-κB (B33B4WP) was purchased from eBioscience and Alexa Fluor® 488 PSMA (FOLH1) was purchased from BioLegend. FITC-CD69 (FN50), Annexin V, and propidium iodide were obtained from BD Pharmingen. Polybrene was purchased from Santa Cruz Biotechnology. Anti-PSMA Chimeric Antigen Receptor T-Cell (CAR-T) Lentivirus, 3rd Generation, scFv-CD28-41 BB-CD34, Pre-packaged Lentiviral Particles was purchased from G&P Biosciences.

Cell Culture: Jurkat cells and PC3 prostate cancer cells were obtained from the ATCC. Jurkat cells and PC3 cells were cultured in the same conditions as previously described.

Primary T cell samples: Primary whole blood samples from prostate cancer patients after informed consent were obtained. T cell isolation and cell culture techniques were carried out as previously described.

T cell activation: Jurkat and primary T cell activation via exposure to FSS was carried out in cone-in-plate viscometers as described previously for 1 hour. In the treatment groups indicated, T cells were incubated with magnetic beads coated with anti-CD3/CD28 antibodies in a 1:1 (T cell:bead) ratio, as indicated by the manufacturer instructions, for 30 minutes prior to FSS exposure.

Flow cytometry antibody staining: The same protocol as described previously was used to stain for intracellular and extracellular proteins.

CAR T cell transduction: Jurkat and primary T cells were activated in combination treatments of FSS and anti-CD3/CD28 beads as described previously, then cultured for 24 hours prior to transduction. For CAR transduction, lentivirus particles containing the viral vector for a third-generation PSMA single-chain variable fragment (scFv) CAR construct (scFv-CD28-41BB-CD34) and polybrene (5 g/mL) was incubated with the T cells for 24 hours. Transduction was carried out as described in the lentivirus manufacturer instructions in 96-well plates and expanded for 7 days. Transduction efficiency was quantified by obtaining the Median Fluorescence Intensity (MFI) of treatment groups in flow cytometry and normalized to a control sample of T cells that was not transduced with the CAR construct.

Cytotoxicity of FSS activated primary T cells/CAR T cells and prostate cancer cells: PC3 prostate cancer cells were seeded at a concentration of 20,000 cells/mL in 24-well plates and incubated with CellTracker™ Deep Red Dye as described in the manufacturer instructions. After 24 hour incubation of PC3s, primary T cells activated using the indicated treatments were added to the cell culture media in a 5:1 (T cell:PC3) ratio immediately following FSS activation. To evaluate the cytotoxicity of CAR T cells, the control group of T cells (not transduced with CAR construct) and CAR T cells activated using the indicated treatments were added to the cell culture media in a 3:1 (T cell:PC3) ratio 7 days following CAR transduction. T cells and CAR T cells were incubated with the PC3s for 24 hours and the Annexin V cell viability assay was performed as described previously.

Annexin V analysis of PC3 cell viability following co-culture with T cells/CAR T cells: The PC3 cell population was isolated for analysis by first gating the high concentration of cells with high forward scatter and side scatter in flow cytometry plots, representing an increase in cell size and granularity, respectively. In contrast, the T cell/CAR T cell population is the cell population characteristic of low forward scatter and side scatter. To further ensure the analysis of only PC3 cells, the forward scatter and side scatter gate was additionally gated based on increased Red-R fluorescence emission compared to a control sample of PC3s not labeled with the Deep Red cell tracking dye. PC3 cells negative for both Annexin V and propidium iodide were identified as being viable. Analysis was done in FlowJo.

Statistics: Results are presented as means and standard deviation (SD) where error bars are indicated. Where statistical significance is indicated, at least three independent experimental trails were performed and an ordinary 2way ANOVA multiple comparison tests was used to compute significance (α=0.05). GraphPad Prism 9 software was used to prepare figures and perform statistical comparisons.

### Example 4. Dendritic Cell Activation is Enhanced with Fluid Shear Stress Stimulation

### Results

### Shear Stress Enhances NF-κB Phosphorylation in DCs at Low Shear Times and Rates.

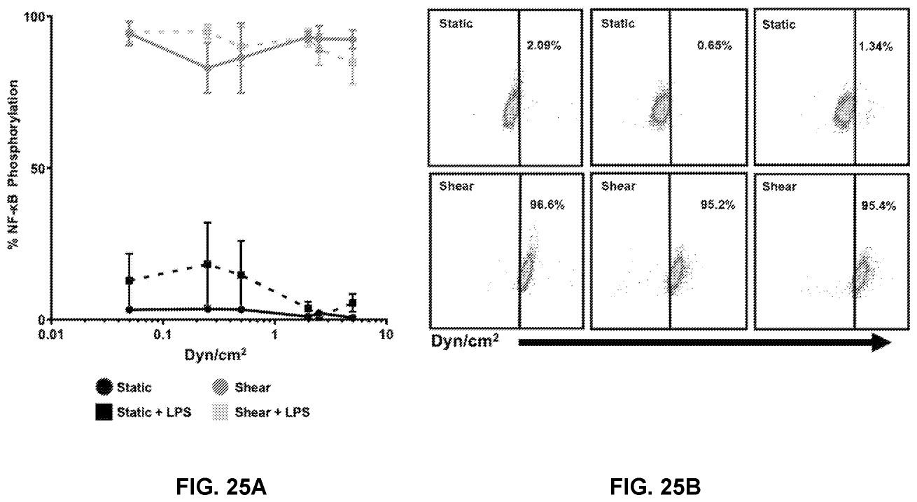

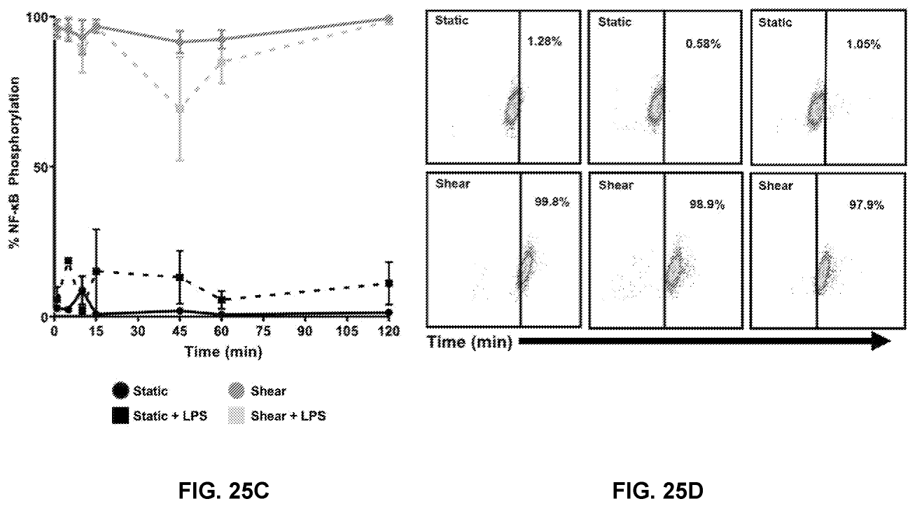

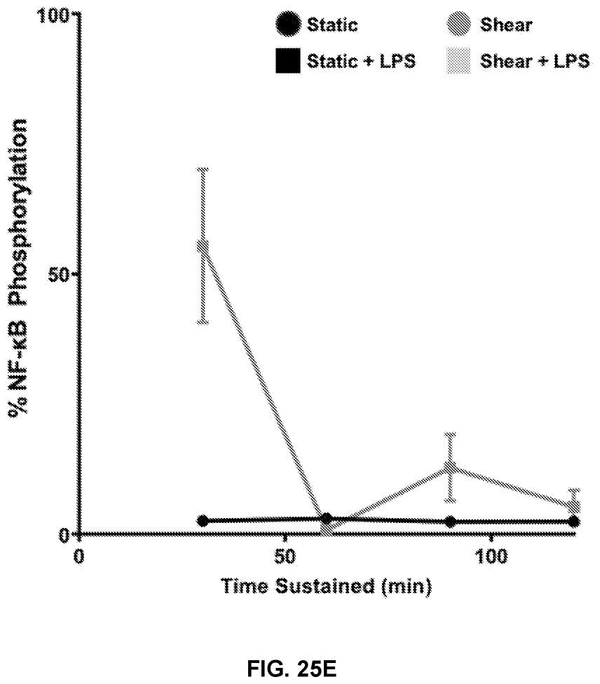

Varying the levels of applied shear stress did not significantly affect NF-κB phosphorylation in DC2.4 cells (FIG. 25A-B). Likewise, varying the time exposed to FSS from 15 min to 2 h was shown to not significantly affect NF-κB phosphorylation (FIG. 25C-D). Even at very low time exposures and shear stress values, enhanced NF-κB phosphorylation was still occurring for cells in shear conditions. NF-κB phosphorylation was shown to be sustained through 30 min (FIG. 25E).

### GsMTx4 Inhibits NF-κB Phosphorylation in DCs

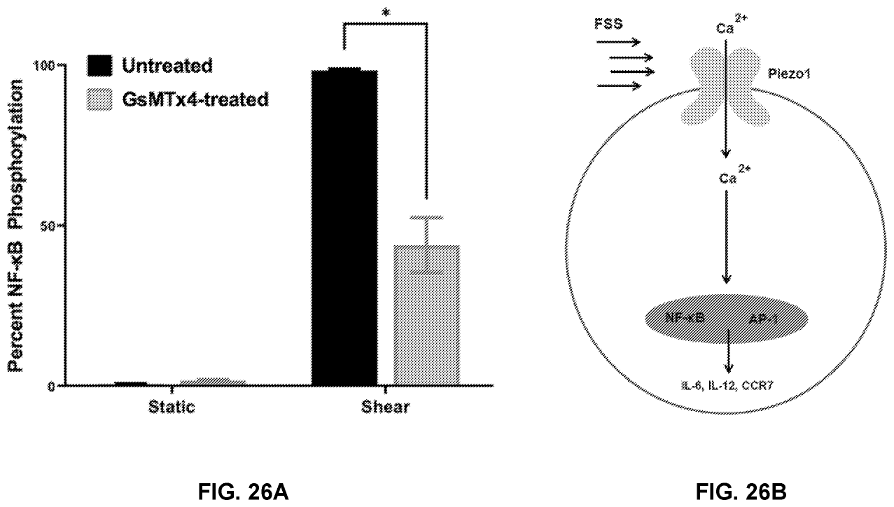

Piezol inhibition via GsMTx4 resulted in reduced NF-κB phosphorylation in DC2.4 cells (FIG. 26A). A mechanism of action was developed to show the significance of activating DCs via FSS resulting in the opening of Piezol to allow for the influx of calcium through the MSC, thereby initiating NF-κB phosphorylation and enhanced AP-1 expression, and ultimately resulting in increased IL-6, IL-12 and CCR7 release (FIG. 26B).

Pre-Coating with Bovine Serum Albumin (BSA) was not the Cause of the Activation Observed with Applied FSS

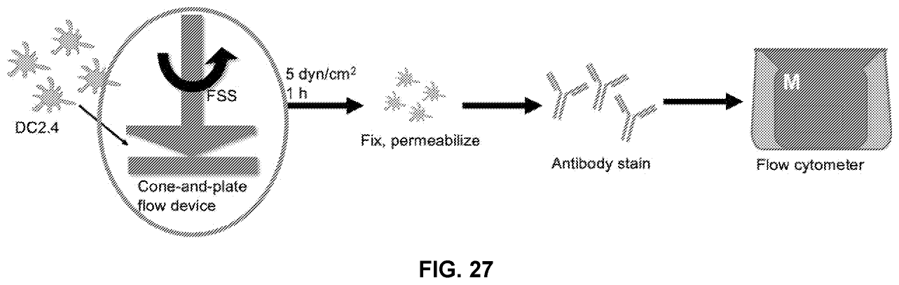

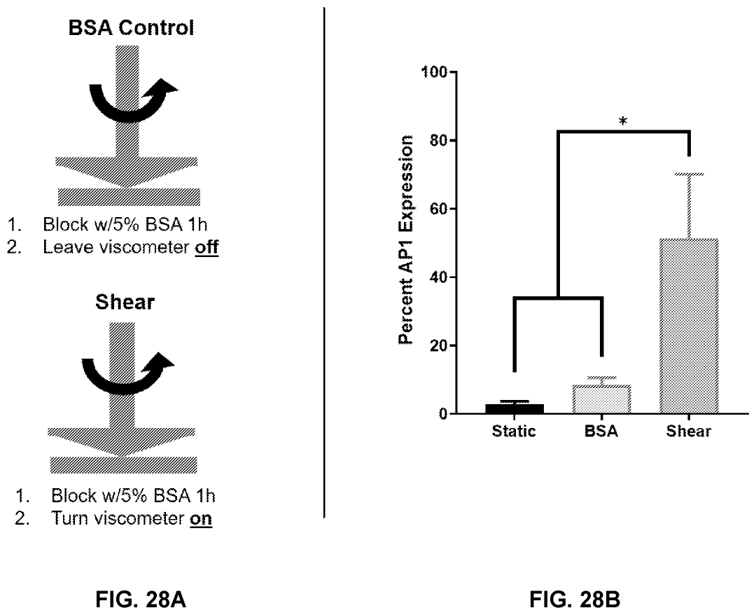

Cone-and-plate viscometers are pre-coated in BSA for blocking, so control experiments were conducted to ensure that BSA was not responsible for activating DCs (FIG. 27) Blocking with BSA alone with no applied FSS did not significantly enhance AP-1 expression compared to FSS, verifying that it is not causing differences between static and shear values (FIG. 28A-B)

### Cytokine Release was Enhanced in DCs Following FSS

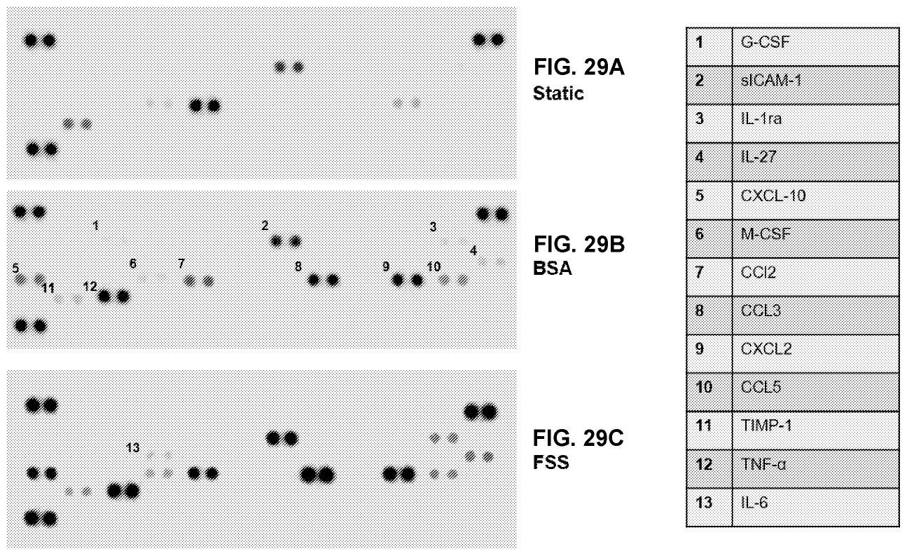

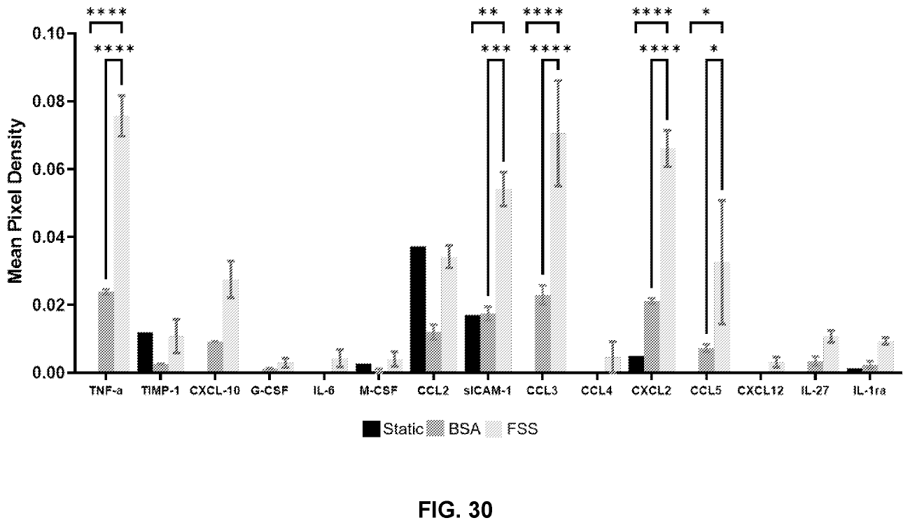

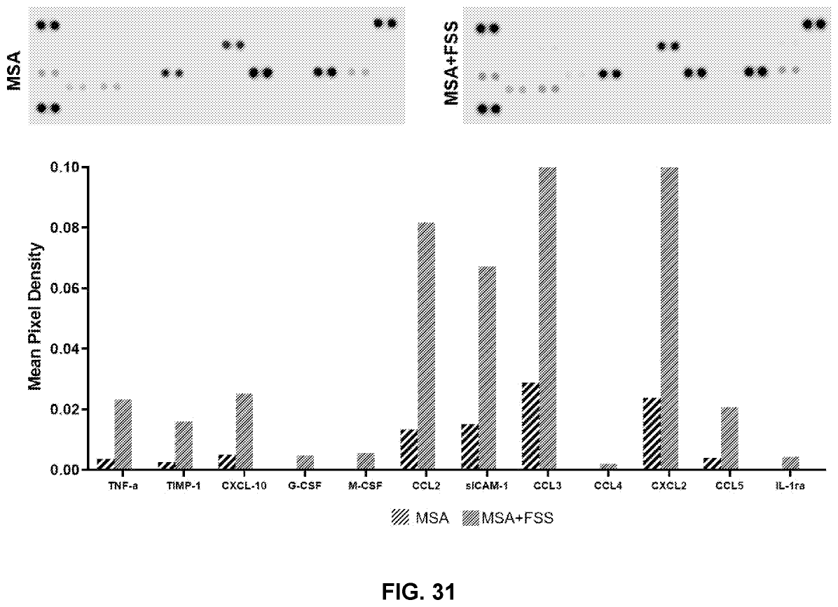

Proteome profiler cytokine array showed increased production of various cytokines following FSS compared to static and BSA (no FSS) controls (FIG. 29A-C). Some of the most significantly upregulated cytokines included TNF-α, sICAM-1, CCL3, CXCL2, and CCL5 (FIG. 30). These effects were similarly observed when pre-coating was performed using mouse serum albumin (MSA), as murine DCs were used in FSS experiments (FIG. 31).

### Trend in Increased Glucose Uptake Following FSS Exposure

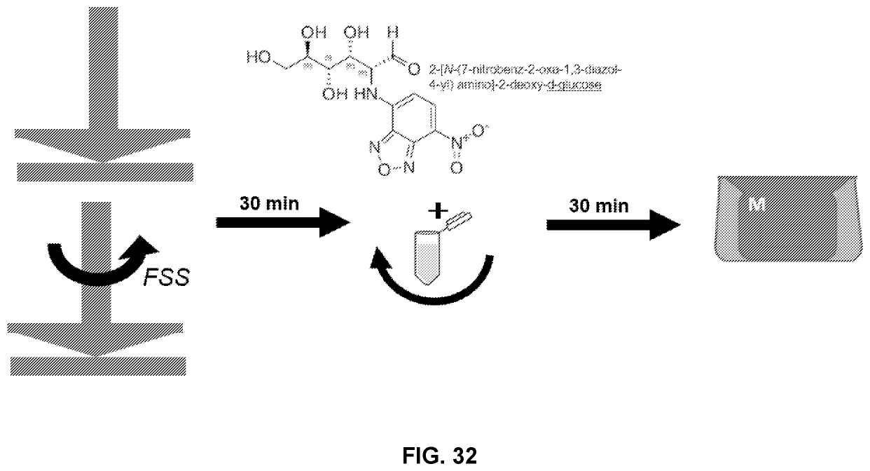

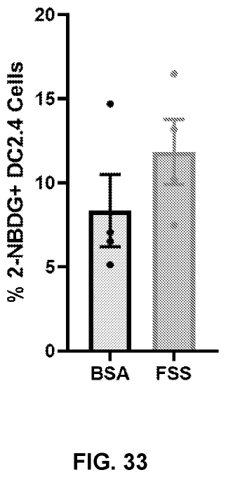

2-[N-(7-nitrobenz-2-oxa-1,3-diazol-4-yl) amino]-2-deoxy-d-glucose (2-NBDG) was used to measured glucose uptake in DC2.4 cells following FSS (FIG. 32). 2-NBDG is a derivative of glucose modified with an amino group. A trend in enhanced glucose uptake was observed following FSS (FIG. 33).

### Activation was Observed in Several Primary DC Cell Lines

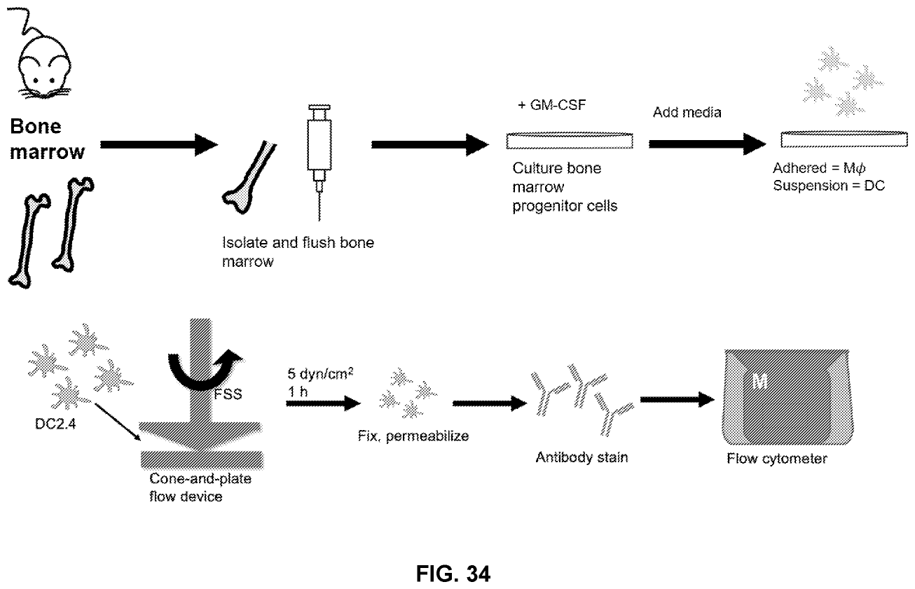

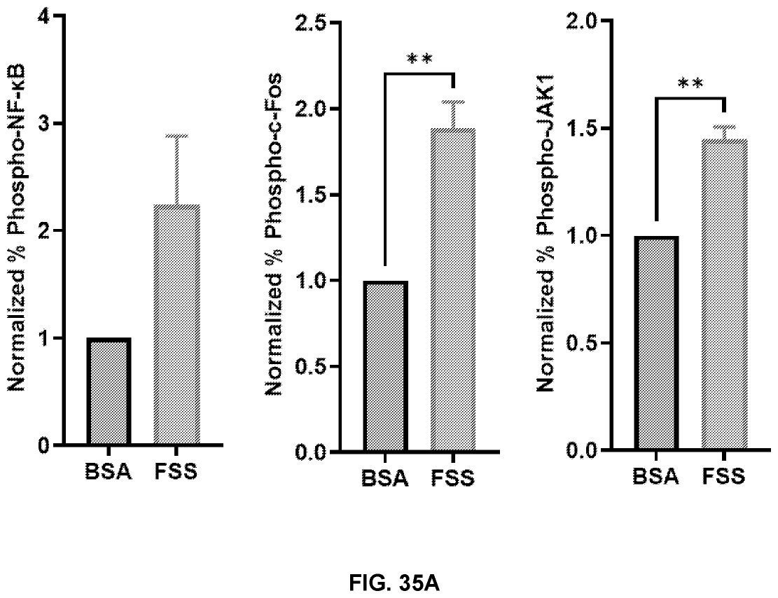

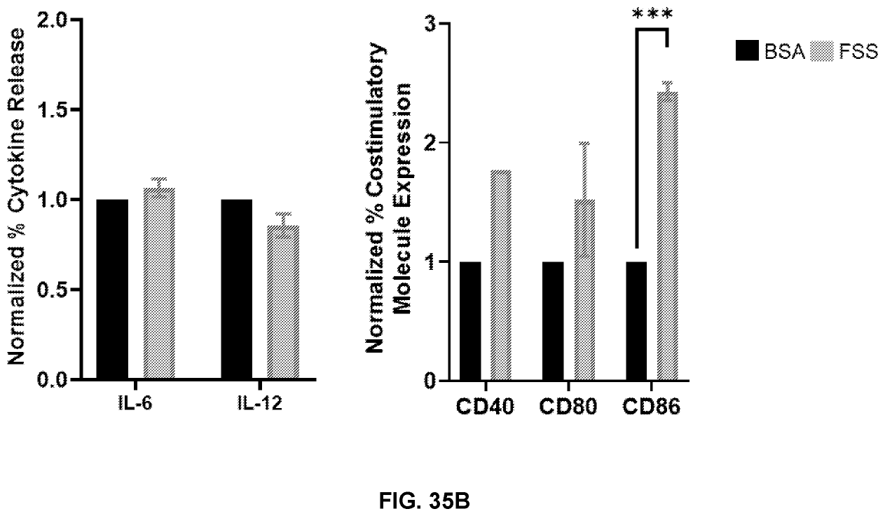

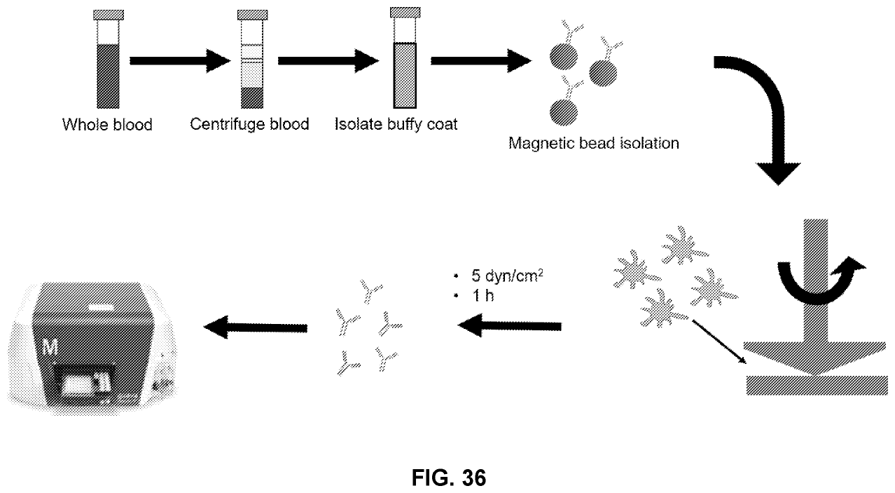

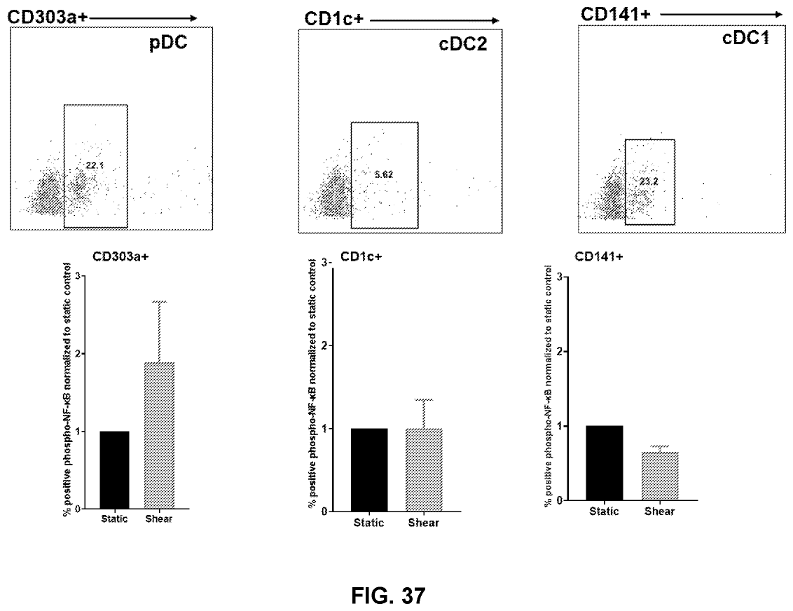

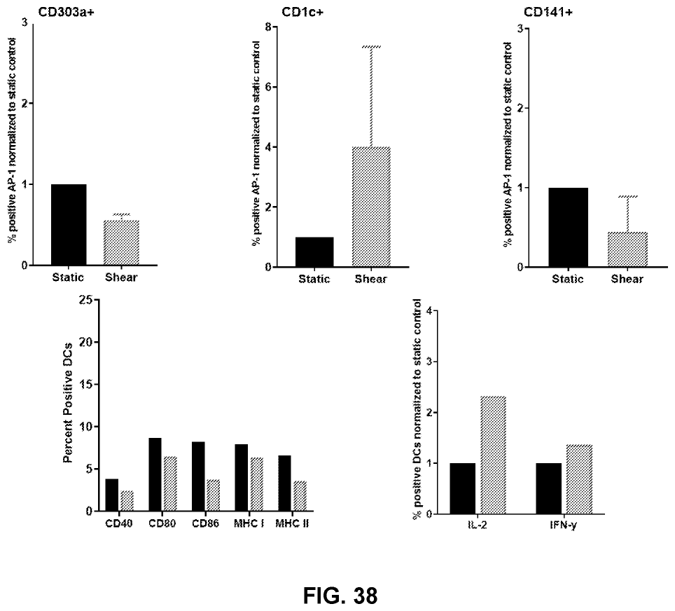

Bone marrow dendritic cells (BMDCs) were isolated from healthy mice (FIG. 34). BMDCs were observed to have upregulated phospo-proteins NF-κB, cFOS and JAK1 and costimulatory molecules expression following 1 h of FSS (FIG. 35A-B). DCs were isolated and analyzed from healthy prostate cancer patients' whole blood (FIG. 36). NF-κB phosphorylation, AP-1 expression, costimulatory molecule expression and cytokine release were enhanced in prostate cancer patient cells following FSS (FIG. 37-38).

### Discussion

In the DC2.4 immortalized cell line, FSS stimulation leads to enhanced phosphorylation of NF-kB and c-FOS, and increased production of cytokines. Even at low shear stress values and short time points, these effects can be observed and sustained. Inhibitors CAPE and GsMTx4 reduce these effects. A trend in enhanced glucose uptake was also observed, giving indicators of metabolic activity following FSS. Significant controls were performed to verify that activation was from FSS and no other factors.

Activation of DCs was also observed in several primary DC lines. In primary mouse BMDCs, FSS stimulation enhanced phosphorylation of NF-kB, c-FOS and JAK1, and increased cytokine release. In prostate cancer patient DCs, FSS stimulation resulted in a trend in increased phosphorylation of NF-kB and c-FOS in specific DC subpopulations, and a trend in enhanced cytokine production.

### Materials and Methods

Varied FSS experiments: Expression of activation markers NF-κB and AP-1 was quantified for DCs activated at varying levels of shear stress and different durations of exposure. For shear stress experiments, cells were exposed for 1 h to shear stress of 0.05, 0.25, 0.5, 2.0 and 2.5 dyn/cm² to analyze different shear stress. For experiments with varying length of shear exposure, cells were exposed to 5.0 dyn/cm² for 1 min, 5 min, 15 min, 45 min, or 2 h. Two replicates were performed for 1 min and 5 min time points, while three replicates were performed for the remaining time points. Analyses were performed as previously described.

Sustained FSS experiments: Since 15 min of FSS stimulation had a comparable effect on NF-κB phosphorylation as other time points, this duration was used for sustained FSS assays. In this experiment, DCs were exposed to FSS for 15 min, and subsequently placed on a rotator for 30 min, 1 h, 1.5 h or 2 h to analyze sustained NF-κB phosphorylation following FSS stimulation. Analyses were performed as described above.

GsMTx4: GsMTx-4 (ab141871) was purchased from Abcam. GsMTx4 was used to inhibit MSCs by inhibiting membrane deformation. Cells were prepared for shear experiments as previously described, then incubated with 10 μmol GsMTx4. Static controls for GsMTx4 were also used. Following FSS, cells were prepared and analyzed for NF-κB phosphorylation as previously described. Two replicates were performed for each condition of this experiment.

BSA control experiments: The setup for this experiment is depicted in FIGS. 27 and 28A, where one group had pre-coating with 2 mL of 5% BSA with the viscometers remaining off (0.0 dyn/cm²) for 1 h and one group had pre-coating with the viscometers turned on to the settings previously described (5.0 dyn/cm2).

Proteome profiler Proteome Profiler Mouse Cytokine Array Kit, Panel A was purchased from R&D Systems. To get a high enough protein concentration, 12 million cells (1 million/mL) underwent each of the conditions: static, BSA (0.0 dyn/cm²), and FSS (5.0 dyn/cm2). Cells were combined in each group, centrifuged and resuspended in 500 μL media and incubated overnight. Following these steps, the protocol in the kit was followed for preparing the array using the cell supernatant after 24 h. LI-COR Odyssey Fc was used to image samples in chemiluminescent for 2 min, and Image Studio Lite was used for image processing.

2-NBDG: DC2.4 cells were washed with complete RPMI and then resuspended in RPMI without glucose at 200,000 cells/mL. 2 mL cell suspension was placed into a viscometer with applied FSS (FSS) and without (BSA). After 30 min, cells were incubated with 1.7 μL of 25 mM 2-NBDG (Abcam) or DMSO as a vehicle control.

BMDC isolation: Cells were isolated and cultured following the standard procedure (Madaan et al., Journal of Biological Methods, 2014. Applied FSS experiments were performed as previously described.

Prostate cancer patient DC isolation: Blood Dendritic Cell Isolation Kit II, human was purchased from Miltenyi Biotec and cells were isolated from 4 mL prostate cancer patient whole blood following the procedure outlined in the kit. Applied FSS experiments were performed as previously described.

### Impact

This study shows similar results for FSS activation of DCs in immortalized cell lines, primary mouse cells and primary human cells. The consistency across cell types shows clinical relevancy and the potential for FSS stimulation to be a successful ex vivo DC therapeutic. Positive results with primary human prostate cancer cells already demonstrates preclinical success of FSS activating DCs with cancer patients.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for manufacturing a human immune effector cell therapeutic comprising:
a) obtaining a population of peripheral blood mononuclear cells (PBMCs) that comprises immune effector cells in a fluid medium;
b) applying from 0.5 dynes/cm² to 20 dynes/cm² fluid shear stress to the immune effector cells for from 5 minutes to 120 minutes; and
c) culturing the activated immune effector cells in a cell growth medium to expand the activated immune effector cells,
thereby manufacturing the human immune effector cell therapeutic.

2. The method of claim 1, wherein the immune effector cell is a T cell, dendritic cell, macrophage, or natural killer (NK) cell.

3. The method of claim 1, wherein step b) further comprises exposing the immune effector cells to one or more activating agents before, during, or after shear stress application.

4. The method of claim 3, wherein the immune effector cell is a T cell.

5. The method of claim 4, wherein the one or more activating agents comprises an anti-CD3 antibody and an anti-CD28 antibody.

6. The method of claim 3, wherein the immune effector cell is a dendritic cell.

7. The method of claim 6, wherein the one or more activating agents comprises lipopolysaccharide (LPS).

8. The method of claim 3, wherein the immune effector cell is a natural killer (NK) cell.

9. The method of claim 8, wherein the one or more activating agents comprises interleukin-2 (IL-2).

10. The method of claim 1, wherein fluid shear stress is applied using a closed loop peristaltic pump system.

11. The method of claim 1, wherein the fluid medium comprises an additive to provide a viscosity of from 0.01 poise to 6.0 poise.

12. The method of claim 11, wherein the additive comprises glycerol, pluronic F68, dextran, polyethylene glycol (PEG), or a combination thereof.

13. The method of claim 1, further comprising assaying the immune effector cells for Piezo1 expression, and selecting a fluid shear stress, viscosity, time, or any combination thereof based on the Piezo1 expression levels.
